(19) United States
(12) Patent Application Publication
(10) Pub. No.: US 2023/0390339 A1
(43) Pub. Date: Dec. 7, 2023

(54) URINE-DERIVED EPITHELIAL CELL CULTURES, NEPHROSPHEROIDS DERIVED THEREFROM AND METHODS OF PRODUCING AND USING SAME

(71) Applicant: TEL HASHOMER MEDICAL RESEARCH INFRASTRUCTURE AND SERVICES LTD., Ramat-Gan (IL)

(72) Inventors: Benjamin DEKEL, Tel-Aviv (IL); Dorit OMER, Yahud-Monosson (IL); Orit HARARI-STEINBERG, RaAnana (IL)

(21) Appl. No.: 18/249,469

(22) PCT Filed: Oct. 19, 2021
(86) PCT No.: PCT/IL2021/051242

§ 371 Date: Apr. 18, 2023

Related U.S. Application Data
(60) Provisional application No. 63/093,332, filed on Oct. 19, 2020.

(51) Int. Cl. A61K 35/22; C12N 5/071; A61P 13/12; G01N 33/50

(52) U.S. Cl. CPC A61K 35/22; C12N 5/0686; A61P 13/12; G01N 33/5082; G01N 2333/165; G01N 2800/52; C12N 2500/40

(57) ABSTRACT

A nephrospheroid comprising urine-derived epithelial cells, the nephrospheroid is capable of forming a tubular nephric tissue upon transplantation. Also provided are methods of producing the nephrospheroid and using same.

### RELATED APPLICATION

This application claims the benefit of priority from U.S. Provisional Patent Application No. 63/093,332 filed on Oct. 19, 2020, which is hereby incorporated by reference in its entirety.

### FIELD AND BACKGROUND OF THE INVENTION

The present invention, in some embodiments thereof, relates to urine-derived epithelial cell cultures, nephrospheroids derived therefrom and methods of producing and using same.

In recent decades, chronic kidney disease (CKD) has become a global epidemic.

Approximately 20 million people in the United States were suffering from CKD in 2014¹ with kidney disease representing the 9^{th} leading cause of death in the United States, killing more than 47,000 Americans in 2013². CKD is a progressive disease, relentlessly advancing from its early stages up to end-stage renal disease (ESRD).

Therapeutic options for CKD are currently limited to supportive care until ESRD ensues, at which time dialysis or renal transplantation are required. Unfortunately, dialysis results in considerable morbidity and mortality, whereas transplantation is limited by the shortage of organs 3 and also requires lifelong immunosuppression. Hence, an effective means of regenerating damaged kidneys is urgently needed.

In recent years, stem/progenitor cell-based therapy has emerged as a potential strategy to replenish damaged kidneys⁴. Ideally, such a strategy should rely on an autologous, readily available cell source. In this regard, several approaches have been proposed, including: 1. Isolation of fetal kidney stem cells. These cells have self-renewal capacity and are multipotent, capable of giving rise to all nephron epithelial lineages. Nonetheless, their isolation involves ethical problems and their allogeneic nature limits their applicability. 2. Directed differentiation of pluripotent stem cells into the renal lineage. Despite much progress in recent years, the end product of this process is usually a mixture of renal cell types, and even non-renal cells⁵. Thus, renal differentiation protocols still require significant improvements before functional kidney tissue can be generated. At the same time, safety issues (e.g. risk of tumorigenesis) still need to be fully addressed before such protocols can be translated into the clinic. 3. Use of adult kidney cells harboring regenerative potential. These, in theory, would represent an autologous cell source, devoid of ethical issues. In this context, the present inventors previously analyzed clonal progeny in the kidney via a transgenic mouse model during steady state and post-injury and showed that new tubular cells arise from lineage-restricted, unipotent precursors that do not cross the boundaries of nephron segments. Thus, the adult kidney replenishes lost cells in a process that involves differentiated cells that self-renew and function locally as heterogeneous lineage-restricted precursors⁶, as opposed to multipotent stem cells.

Accordingly, it was found that human kidney epithelial cells, derived from nephrectomized adult kidneys (KD-EpC), are capable of forming three dimensional (3D) spheroids (termed nephrospheres; nSPH) in serum free medium (SFM)⁷ (US20130059325). When cells collected from nSPH are grafted onto the chorioallantoic membrane (CAM) of the chick embryo, they generate renal tubules, a trait that is reduced or lost when applying the 2D expanded KD-EpC. Very recently⁸, it was demonstrated that nSPH formation involves the activation of a genetic program that recapitulates nephrogenesis, including mesenchymal-epithelial and proliferation-quiescence transitions, alongside activation of tissue-specific genes, such that region-specific renal lineages are represented within a growing nSPH. Importantly, it was found that human nSPH-derived cells were capable of generating renal epithelial tubules and exerting an anti-fibrotic effect, resulting in a beneficial effect in a mouse model of CKD⁸.

The isolation and culturing of human urine-derived cells has been first described several decades ago^{9,10}, although the established cultures were poorly defined. Later reports were able to isolate better characterized populations of renal cells. These include podocytes, which were abundant in the urine of patients with glomerular diseases but rare in normal individuals, and in both cases, quickly underwent senescence.

A second population isolated from urine samples is an MSC-like population, harboring the typical surface marker expression pattern and the multilineage differentiation potential into mesenchymal lineages^{11,12}. While being easily obtainable from various sources, including the kidney¹³, and potentially relevant for studying diseases arising from renal MSCs, such as the kidney tumor angiomyolipoma¹⁴, the MSC population in the kidney is devoid of renal regenerative potential, as previously discussed^{3,15} MSCs have a role in renal fibrosis and tumorigenesis, however, they do not have the potential to differentiate into renal lineage. That was proven by a model of lineage tracing that showed that nascent renal tubular cells following ischemic injury obtained from the renal epithelium, rather than exterior origin¹⁶.

Lastly, several reports described the isolation and culturing of tubular epithelial cells from urine sample, using collagen-coated plates, which included both proximal and distal tubular cells ^{17,18}, however subculturing the cells was difficult, and possible only for proximal tubular cells¹⁸, which limited their use for therapeutic purposes. By growing the cells between two layers of collagen gel, Inoue et al. were able to derive 3D tubule-like structures composed of CD13⁺ PT cells, which have the potential to serve as a cellular model of PT cells. Nonetheless, formation of tubular structures of the distal phenotype were not reported, nor was their tubulogenic potential, or possible therapeutic effect assessed in an in-vivo model. These reports exemplify the fact that upon in-vitro culturing, renal tubular cells rapidly lose their epithelial identity, and cannot function as tubulogenic precursors.

It is also envisaged that such cell systems can be used as research models for studying human disease. The spread of the severe acute respiratory syndrome coronavirus 2 (SARS-CoV-2) has resulted in a global health crisis, leading to over 2,500,000 cases of coronavirus disease (COVID-19) and 170,000 deaths as of Apr. 20, 2020. Aside from supportive care, no treatment is available, with mortality rates estimated at about 3%¹⁹. Importantly, SARS-CoV-2 is significantly more contagious than its predecessors, SARS-CoV and MERS-CoV. It was previously shown that a key step in the cell entry of SARS-CoV-2 involves association of the Coronavirus spike glycoproteins to the membrane-bound ACE2 and TMPRSS2^{20,21}, as well as potentially to the recently described BSG²².

Nonetheless, there are still significant knowledge gaps regarding the cellular mechanisms involved in the infection of the virus, impeding the road to treatment. One of the major obstacles in the elucidation of these mechanisms is the lack of a reliable primary human cell model. Such a model should ideally be 1. Readily available; 2. Devoid of genetic manipulations (e.g. gene over-expression); 3. Obtainable from a wide range of patients (as opposed to cell lines); and 4. Abundantly express ACE2. Importantly, primary airway cells, which are the main target of the SARS-CoV-2 virus, are difficult to obtain, requiring invasive procedures.

Therefore there is high need for a more available cell source, which could facilitate the establishment of a human cell model for COVID-19.

Interestingly, aside from the respiratory system, ACE2 has been shown to be highly expressed in the epithelium of the human kidneys (particularly proximal tubules)²³.

Additional background art includes:

WO2011/141914 disclosing methods of generating a nephrospheroid comprising culturing human adult kidney cells in a culture medium under non-adherent conditions.

### SUMMARY OF THE INVENTION

According to an aspect of some embodiments of the present invention there is provided a method of expanding kidney epithelial cells, the method comprising:
(a) isolating cells from urine of a subject;
(b) culturing the cells under adherent conditions, so as to obtain urine-derived epithelial cells (UD-EpC);
(c) passaging the UD-EpC.

According to some embodiments of the invention, the culturing is performed in a serum-free medium.

According to some embodiments of the invention, the culturing is performed in RE:MC.

According to some embodiments of the invention, the culturing is performed in the presence of CD40 ligand (CD40L), e.g., (2-10 ng/ml each).

According to some embodiments of the invention, the culturing is performed in the presence of neuregulin-1 (NRG1), e.g., (5-10 ng/ml).

According to some embodiments of the invention, the culturing is performed in the presence of isolated mitochondria.

According to some embodiments of the invention, the isolating is by centrifugation.

According to some embodiments of the invention, the adherent conditions comprise gelatin coating.

According to some embodiments of the invention, the subject is a male subject.

According to some embodiments of the invention, the subject is a female subject.

According to some embodiments of the invention, the passaging is performed to enrich UD-EpC and deplete squamous epithelial cells of vagina and/or bladder origin).

According to some embodiments of the invention, the subject is a healthy subject.

According to some embodiments of the invention, the subject is diagnosed with a kidney disease.

According to some embodiments of the invention, the kidney disease is a chronic kidney disease (CKD).

According to some embodiments of the invention, the subject is a human subject.

According to some embodiments of the invention, the human subject is an adult.

According to some embodiments of the invention, the UD-EpC express Ace2.

According to some embodiments of the invention, the UD-EpC are CD13+/EMA+/EpCAM+.

According to an aspect of some embodiments of the present invention there is provided a culture comprising the UD-EpC obtainable according to the method.

According to some embodiments of the invention, is characterized by gene expression as in FIG. 8C (human urine), e.g., higher expression of ATP12A, ACMS2A and/or SLC16A7 than that derived from human kidney.

According to an aspect of some embodiments of the present invention there is provided a method of producing a nephrospheroid, the method comprising culturing the UD-EpC of the method under non-adherent conditions, thereby generating the nephrospheroid.

According to some embodiments of the invention, the nephrospheroid is capable of forming a tubular nephric tissue upon transplantation.

According to some embodiments of the invention, the nephrospheroid is capable of generating a proximal tubule compartment.

According to some embodiments of the invention, the proximal tubule compartment expresses Ace2.

According to some embodiments of the invention, the nephrospheroid is capable of generating a distal tubule compartment.

According to some embodiments of the invention, the nephrospheroid is CD13+/EMA+/EpCAM+/Ace2+ at the protein level and CD13+/EMA+/EpCAM+/Ace2− at the RNA level.

According to an aspect of some embodiments of the present invention there is provided a nephrospheroid obtainable according to the method.

According to an aspect of some embodiments of the present invention there is provided a nephrospheroid comprising urine-derived epithelial cells, the nephrospheroid is capable of forming a tubular nephric tissue upon transplantation.

According to an aspect of some embodiments of the present invention there is provided cells or secretome of the nephrosphere.

According to an aspect of some embodiments of the present invention there is provided a method of regenerating renal function, the method comprising administering to a subject in need thereof the nephrospheroid of any one of claims 26-27 or cells or secretome of claim 28, thereby regenerating renal function.

According to an aspect of some embodiments of the present invention there is provided a method of drug design, the method comprising determining an effect of a test drug on the nephrospheroid of any one of claims 26-27 or cells or secretome of claim 28.

According to some embodiments of the invention, the determining is performed in the presence of a Coronavirus.

According to some embodiments of the invention, the Coronavirus is SARS-CoV-2.

According to an aspect of some embodiments of the present invention there is provided a method of analyzing infectivity of a Coronavirus, the method comprising:
(a) contacting a renal epithelial cell culture or a nephrospheroid produced of the culture with a Coronavirus; and
(b) determining infectivity of the Coronavirus in the culture or in the nephrospheroid following the contacting.

According to some embodiments of the invention, the renal epithelial cell culture is kidney-derived or urine-derived epithelial cells.

According to an aspect of some embodiments of the present invention there is provided a method of personalized therapy, the method comprising:
(a) contacting a Coronavirus-infected renal epithelial cell culture or nephrospheroid produced of the culture with a test drug; and
(b) determining an alleviation in viral load following the contacting, the alleviation being indicative of an efficacious therapy.

According to some embodiments of the invention, the renal culture or nephrospheroid is autologous.

According to a specific embodiment, the nephrospheroid has an anti-fibrotic activity.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E: (A) Scheme illustrating the establishment of a protocol for generating UD-EpC-nSPH: Midstream urine samples from each donor were collected for four consecutive days. Following centrifugation, cells were isolated and seeded into doublets of three gelatin-coated wells, each containing one of three media: SCM, KSFM:PR or RE:MC, thereby establishing P0. In order to maximize the number of epithelial cells, two days after seeding P0 the upper liquid (UL) of each well was collected, centrifuged and reseeded onto a new gelatin-coated well with the same, fresh medium; these cultures are termed the UL wells. 14-21 days later, upon reaching confluence, P0 cells were passaged and grown in the same medium, as P1. Then, P2 or P3 cells were passaged into low-adherence plates, thereby generating nephro-spheres. (B-E) Percentage of urine samples that generated P0 cultures: cells that reached P0, cells that reached P1, gender and age.

FIGS. 2A-E: (A) Representative morphology of UD-EpC cultured in each of the three media types along P0-P2 passages. (B) Squamous Cells found in P0 cultures, mostly of female donors. (C) UD-EpC cultured in SFM (UD-EpC-SFM), showing failure to expand. (D) Number of cells generated in P0 cultures from 100 ml of urine according to medium type. UD-EpC, urine-derived epithelial cells; SFM, serum-free medium. (E) Flow cytometry analysis comparing the expression levels of the proximal tubule marker CD13, distal tubule marker EMA and epithelial marker EpCAM, in UD-EpC cultured in each of the media types.

FIGS. 3A-E: (A) Representative morphology of cultured UD-EpC derived from CKD patients (CKD-UD-EpC), grown in RE:MC or KSFM:PR (B) Number of cells generated in P0 and P1 cultures from 100 mL of urine according to medium type. (C) Flow cytometry analysis comparing the expression levels of the proximal tubule marker CD13, distal tubule marker EMA and epithelial marker EpCAM, in UD-EpC cultured in each of the media types in both glomerular and tubular diseases. (D) Expression analysis of human nephron segment specific markers in UD-EpCs cultured with KSFM in comparison to REMC (E) Gene set enrichment analysis (GSEA) showing down regulation of cell cycle progression related gene sets in UD-EpCs cultured with KSFM in comparison to REMC.

FIGS. 4A-D (A) Venn diagram represents intersection between highly expressed genes in UD-EpCs and growth factor receptors genes (GFRs). Genes that were considered as highly expressed are the top 10% most highly expressed genes according to average TPM values in UD-EpCs, GFRs list was determined by uniprot. (B) TPM values of CD40LG and NRG1 receptors in UD-EpCs. (C) UD-EpC treated with NRG1 supplement showed higher absorbance in proliferation assay (MTS), reduction in doubling time, resulted in higher cell yield (significantly at 5 ng/ml). (D) UD-EpC treated with CD40L supplement showed higher absorbance in proliferation assay (MTS), reduction in doubling time, resulted in higher cell yield (significantly at 5 ng/ml).

FIGS. 5A-C: (A) UD-EpC that derived from healthy, or CKD donors have the capacity to generate nephrospheroids (nSPH), regardless the media they've been culture with. The nSPH can generated from adherent cells at passage 1-3. (B) Immunostaining of kidney epithelial markers: cytokeratin, CD13 and EMA. (C) FACS analysis of kidney epithelial markers of nSPH originated from glomerular or tubular CKD, cultured with KSFM or REMC (when grown in 2D conditions).

FIGS. 6A-E: (A) Euclidean distance analysis was performed on RNA-seq data to illustrate variation in transcription levels between samples. The difference between cell types is demonstrated by a heatmap. While UD-EpC are different from UD-nSPH, samples originated from different donors or cultured with different media are more similar to each other and cluster together. There is greater similarity between. Darker color indicates greater similarity. (B) Principal component analysis was performed on RNA-seq data to illustrate variation in transcription levels between samples. (C) Volcano plot representation of genes deferentially expressed in UD-nSPHs in comparison to UD-EPCs. Light-grey points denote differentially expressed genes (padj<0.05, Log 2(fold-change)>1). Genes that either promote or inhibit ETM are labeled. While genes that activate EMT are down-regulated in UD-nSPH (cyan), genes that inhibit EMT are up-regulated (red). (D) Gene set enrichment analysis (GSEA) showing up regulation wnt signaling in UD-nSPH in comparison to, and down regulation of cell cycle progression as well as EMT. (E) Heatmap of nephron segment markers shows upregulation of these genes in UD-nSPH in healthy and sick donors.

FIGS. 7A-C: (A) RNA-seq analysis of proximal tubule markers in samples derived from CKD patients in comparison to samples derived from a healthy donor. Log 2 fold change ratio between healthy and CKD donor of all proximal tubule marker in UD-EpC (red) and in UD-SPH (cyan). Each dot represent one gene. While in cells derived from a patient with a chronic renal failure disease (HU21) we could not detect a difference in the expression level of proximal tubule markers in both UD-EpC and UD-SPH, in samples derived from patients with Fanconi syndrome (HU22, HU23) a decreased expression in UD-EpC was detected but was back to normal in UD-SPH. (B) Table of proximal marker genes. Yes represent genes up-regulated in UD-SPH in comparison to UD-EpC and down-regulated in UD-EpC of CKD patients in comparison to UD-Epc derived from a healthy donor (C) TPM values of selected proximal tubule markers in UD-EpC (light blue) and UD-SPH (Blue) in samples derived from a healthy donor (HU18) from a patient with a chronic renal failure disease (HU21) and patients with Fanconi syndrome (HU22, HU23). While the TPM values of these markers were lower in EpC derived from Fanconi syndrome patients, they were elevated in UD-SPH and were similar to HU18 and HU22.

FIGS. 8A-C: (A) Venn diagram showing DE genes in transcriptome data of both KD-nSPH and UD-nSPH of downregulated and upregulated genes in comparison to their adherent counterparts. (B) Pearson correlation of samples derived from adult kidney tissue and urine origins. (C) Expression values of nephron segment genes is higher in samples derived from urine, both 2D and 3D.

FIGS. 9A-E: (A) RNA-seq showed expression levels of TMPRSS2, BSG (CD147), ANPEP and ACE2 in UD-EpC and KD-EpC (2D) compared to nSPH (3D). B. ACE2 and CD147 immunostaining of fetal kidney (FK) and adult kidney (AK) tissue. C. ACE2 and CD147 immunostaining of UD-nSPH from healthy donor and CKD patient (adult and child). D. Double IF staining of ACE2 and the proximal marker LTL in UD-nSPH. (E) infection of hCOV-229E in urine and kidney derived cells and lung cells as a positive control.

FIGS. 10A-C: (A) pfuE/ml of SARS-CoV-2 in the medium of UK/UD-EpC, UK/UD-nSPH infected with the virus. (B) Relative expression of type I interferon response-related genes in infected hKEpC vs. kSPH. (C) Treated kSPH with increasing doses of the ACE inhibitor Ramipril for 48 hours analyzed by qPCR to test the effect on the expression of ACE2, TMPRSS2, and BSG.

FIGS. 11A-C: (A) Scheme illustrating the assay used to test the in-vivo differentiation potential of UD-nSPH. The latter were collected and injected within Matrigel into the subcutaneous tissue of NOD-SCID mice, where they were allowed to form vascularized grafts for 2-3 weeks, after which the grafts were removed for histological analysis. (B) Immunofluorescent staining of grafts generated from UD-nSPH, demonstrating the formation of tubular structures, expressing the human specific marker HLA, proximal and distal tubule markers, CD13 and EMA, respectively. Some of the tubular structures demonstrate a patent lumen. The tubule-like structures obtained from nSPH were stained for the transporter markers: AQP1 (proximal) and SLC12a3 (distal). Shown are representative stainings for these markers. (C) same procedure with UD-nSPH isolated from CKD patients, demonstrate same capacity of healthy donors.

FIGS. 12A-C: (A) Scheme describing the experimental protocol. (B) Blood creatinine and urea, urine protein and creatinine clearance, showing decrease in kidney function. (C) Blood electrolytes measurements supporting the kidney function decrease.

FIGS. 13A-C: (A) HLA staining to trace the injected cells in the host mouse kidney, showed engraftment of the human cells into the mouse parenchyma. (B) The engrafted cells stained for the kidney epithelial markers EMA and CD13. (C) Masson-Trichrome staining for fibrosis detection showed fibrotic areas in control mouse tissue comparing the treated tissue. quantification of the blue pixels (fibrotic tissue) showed higher number in the control mouse tissue.

FIGS. 14A-E (A) Scheme of co-culture of UD-EpC or UD-SPH with fibroblasts. (B) Gene expression of fibroblasts that were co-cultured with UD-EpC showed up-regulation of collagens and periostin compared to fibroblasts that were co-cultured with UD-nSPH (C) that showed downregulation in fibrosis related genes. (D) Transcriptome of UD-nSPH shows upregulation of anti-fibrotic genes compare to UD-EpC that shows upregulation in profibrotic genes. (E) Secretome analysis by proteomics shows that UD-nSPH secrete reno-protective molecules and anti-fibrotic molecules.

### DESCRIPTION OF SPECIFIC EMBODIMENTS OF THE INVENTION

The present invention, in some embodiments thereof, relates to urine-derived epithelial cell cultures, nephrospheroids derived therefrom and methods of producing and using same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

While relying on kidney epithelial cells (KD-EpC) as the basis for renal regenerative strategies is theoretically possible, it is significantly limited by the need for carrying out a renal biopsy, which is invasive, prone to complications, and limited in the number of cells that can be obtained.

Herein, the present inventors were interested in determining whether urine-derived epithelial cells (UD-EpC) can replace KD-EpC as the starting material for establishing nSPH with regenerative potential. Considering that approximately 68,000-78,000 epithelial cells per hour are excreted in the urine²⁴, and that urinary cells have been previously cultured in-vitro^{11,12,18}, the present inventors reasoned that urine samples could represent a non-invasive source of cells. Indeed, the present inventors successfully established cultures of human UD-EpC and identified specific culture conditions allowing their expansion. Moreover, the present inventors show that UD-EpC are equivalent to KD-EpC in their ability to form nephrospheres (nSPH) with tubulogenic capacity. Importantly, UD-EpC derived from CKD patients, were as effective in generating nSPH, as their counterparts derived from healthy individuals. In addition, the present inventors show that the UD-nSPH abundantly express the SARS-CoV-2 receptors ACE2 and TMPRSS2 and accordingly readily infected by the virus, which makes them a unique and readily-available personalized cellular model for SARS-CoV-2 infection in humans.

Thus, according to an aspect of the invention, there is provided a method of expanding kidney epithelial cells, the method comprising:
(a) isolating cells from urine of a subject;
(b) culturing said cells under adherent conditions, so as to obtain urine-derived epithelial cells (UD-EpC);
(c) passaging said UD-EpC.

As used herein “expanding” refers to enriching and increasing the number of cells from one passage to another. Typically the cells are cultured for less than three passages, more preferably for less than two passages. (P0, P1 till P2) without losing the kidney cell phenotype, such as determined by marker expression, e.g., CD13, EMA and EpCAM and renal tubular potency. According to a specific embodiment, there is at least 5 or 10 fold increase from P0 to P1 and at least 5 or 10 fold increase from P1 to P2.

Antibodies for the above mentioned cell markers are commercially available. Examples include but are not limited to, EPCAM (MiltenyiBiotec), EMA (Cell-marque), CD13 (ABCAM).

As used herein, the term “enriching” refers to a procedure which allows the specific subpopulation of renal cells to comprise at least about 50%, preferably at least about 70%, more preferably at least about 80%, about 95%, about 97%, about 99% or more renal stem cells having the desired signature (e.g. EpCAM+, CD13+, EMA+).

The enriching may be effected using known cell sorting procedures such as by using a fluorescence-activated cell sorter (FACS).

As used herein, the term “flow cytometry” refers to an assay in which the proportion of a material (e.g. renal cells comprising a particular maker) in a sample is determined by labeling the material (e.g., by binding a labeled antibody to the material), causing a fluid stream containing the material to pass through a beam of light, separating the light emitted from the sample into constituent wavelengths by a series of filters and mirrors, and detecting the light.

A multitude of flow cytometers are commercially available including for e.g. Becton Dickinson FACScan and FACScalibur (BD Biosciences, Mountain View, CA). Antibodies that may be used for FACS analysis are taught in Schlossman S, Boumell L, et al, [Leucocyte Typing V. New York: Oxford University Press; 1995] and are widely commercially available.

Another method of cell sorting is magnetic cell sorting which can be used according to the knowledge of the skilled artisan. Other methods are available too.

It will be appreciated that the enriching may also be effected by depleting of non-relevant subpopulations. Notably, initial urine samples comprise, aside from epithelial cells, also squamous cells (mostly observed in samples derived from women, likely of urethral origin), leukocytes and semen, in accordance with previous reports²⁵. It well be appreciated that these cell types do not adhere to the plate surface and gradually disappear following a few medium aspirations.

Thus, in effect the passaging is performed to enrich UD-EpC and deplete squamous epithelial cells of vagina and/or bladder origin.

Therefore at P0 or P1 the culture is pure, i.e., more than 90%, 95% or 99% cells are kidney epithelial cells.

Once isolated, cells of the present invention may be cultured and their phenotype (e.g., function, morphology, marker(s)) may be further analyzed as described below.

In order to isolate the cells from urine, mid-stream urine samples are collected from male or female subjects, e.g., human or non-human, preferably human, at any age e.g., adult—above 18 years. The subject can be healthy or diagnosed with a kidney disease, such as chronic kidney disease (CKD), such as representing inherited or acquired glomerular and/or tubular and/or tubulo-interstitial disorders: glomerulonephritis, glomerulosclerosis, interstitial nephritis, any form of a ciliopathy, renal tubular acidosis (RTA), Pearson syndrome, Fanconi syndrome, proximal tubulopathy, renal tubule dysgenesis (RTD), Bickel-Fanconi, any form of congenital anomalies of the kidney and urinary tract (CAKUT).

According to a specific embodiment, isolating urine cells is by centrifugation. Thus, according to a specific embodiment, to obtain kidney cells from the urine, urine samples are centrifuged (e.g., 400×g), washed with buffer e.g., PBS and centrifuged, such as at higher, same or lower gravity e.g., at 200×g, than the first round. Then the cell pellet is seeded on adherent substrate.

According to a specific embodiment, the adherent conditions comprise gelatin coating, though other adherent natural or synthetic matrices may be used.

The phrase “adherent conditions” refers to conditions in which the cells attach to the surface of a container in which they are cultured such that a substantial portion of the cells cannot be removed from the surface of the container by mechanical manipulations that do not cause significant damage to the cells.

The conditions for culturing urine epithelial cells further include serum (for instance, RE:MC comprises serum).

As used herein, “serum” refers to naturally occurring serum or serum replacement. The use of serum replacement may be beneficial to avoid xeno-contamination.

When cells reach confluency (P0) they are harvested such as by the use of a protease, e.g., trypsin and seeded to the next passage (P1).

Cells grown in 2D are termed “UD-EpC”. According to some embodiments the cells are grown in the same medium in the later passages (e.g., P1, P2 . . . ) or medium can be changed according to the knowledge of the skilled artisan as long as the selected medium is such that allows expansion without affecting the differentiation potential. P0 can be 14-21 days, P1 can be 4-12 days and P2 can be 5-10 days.

UD-EpC are characterized by a distinctive morphology and growth characteristics, such as cuboidal epithelial morphology and adherence.

According to a specific embodiment, RE:MC or KSFM media are used for establishing UD-EpC cultures.

According to a specific embodiment, RE:MC medium is used for establishing UD-EpC cultures.

The medium may change dependent on the starting population i.e., healthy or disease derived, such as described in the Examples section.

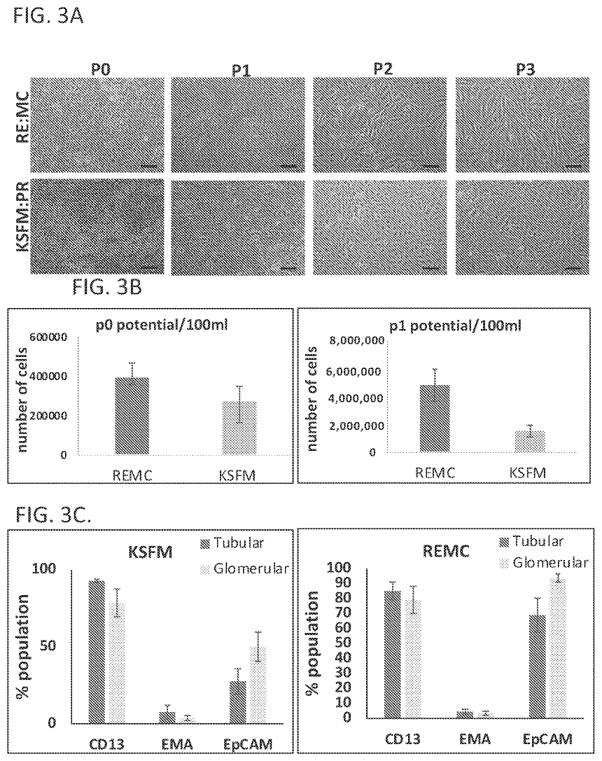

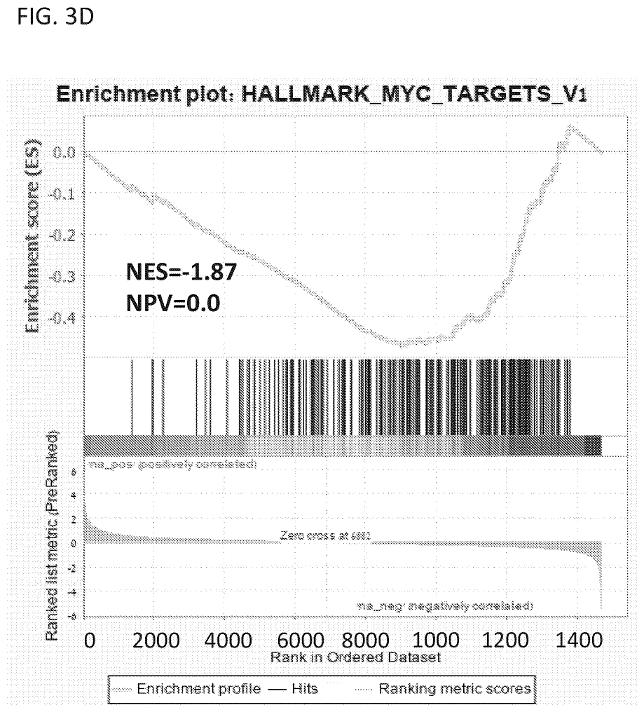

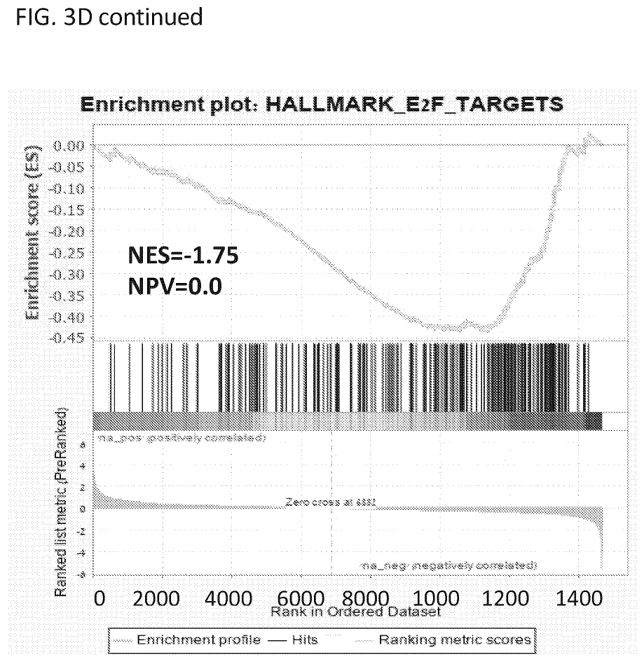

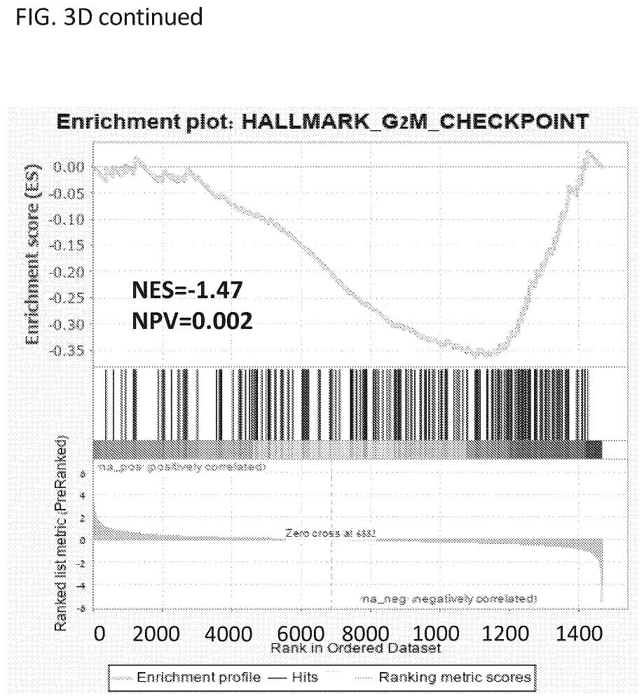

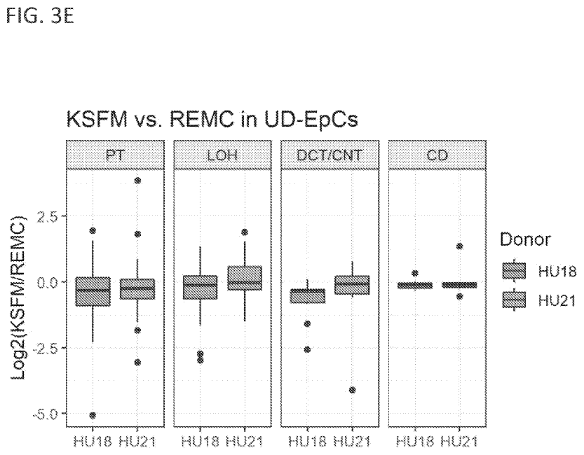

Specifically, according to some embodiments, when using RE:MC, UD-EpC derived from diseased patients exhibit a higher cell yield, compared to KSFM. In other embodiments, UD-EpC from patients with tubular vs. glomerular diseases, is advantageous in growing the cells in RE:MC as it provides a mostly epithelial phenotype of the cells, as evident by EpCAM expression (FIG. 3C). In contrast, culturing the cells in KSFM resulted in a significantly lower percentage of epithelial cells, especially in the case of tubular diseases when considering the expression levels of the epithelial marker EpCAM. In both media types, the majority of cells were of the proximal phenotype, as evident by the expression of CD13 and EMA (FIG. 3C).

According to a specific embodiment, culturing is performed in the presence of CD40 ligand (CD40L), e.g., (2-10 ng/ml each).

According to a specific embodiment, culturing is performed in the presence of neuregulin-1 (NRG1), e.g., (5-10 ng/ml).

Factors are commercially available such as: CD40L (Peprotech) and NRG1 (Peprotech).

The addition of such factors to the growth medium significantly improves cell viability such as determined via the MTS (Methyltetrazolium salt proliferation assay) assay, reduces the doubling time and results in a higher cell yield such as by at least 1.5 fold, as compared to the absence of such factors.

According to a specific embodiment, culturing is performed in the presence of isolated mitochondria. The data shows that culturing the urine derived cells with exogeneous mitochondria results in enhanced mitochondrial activity of urine derived cells potentially leading to enhanced renal potency.

In terms of marker expression the UD-EpC are EpCAM+, CD13+ and optionally EMA+. Additional markers include, but are not limited to, C24, ATP1A1, EMP3 and/or CLDN1.

According to an aspect of the invention, there is provided a culture comprising the UD-EpC obtainable according to the as described herein.

It will be appreciated that the ability to form nephrospheroids from urine is surprising as the UD-EpC should maintain their renal properties during the transfer from the kidney to the bladder which involves a harsh chemical and physical environment. Specifically, the urine is a hostile environment for cells and hence is a difficult to culture cell source. Embodiments of the invention first employ a method to isolate and expand renal cells from the urine in 2D. This allows to up-scale numbers of cells. Importantly, while doing so, renal identity and renal potency is lost in culture. This is circumvented that by shifting 2D growth to 3D growth and generation of kidney spheroids from the 2D cultures. The established kidney spheroids now harbor cells that regain renal identity and renal potency.

Thus, according to an aspect of the invention, there is provided a method of producing a nephrospheroid, the method comprising culturing the UD-EpC under non-adherent conditions, thereby generating the nephrospheroid.

As used herein, the term “nephrospheroid” refers to a 3 dimensional spherical or aggregate of kidney cells. Also referred to as “kidney spheroid”. It may also be referred to as a tubular organoid. The nephrospheroid comprises at least two cell types and is not of a one single cell clonal origin. The nephrospheroid is capable of regenerating renal structures such as tubular structures that are retained long term in host kidney and exerts beneficial effects on microenvironment and reduces fibrosis and inflammation. By the use of the term nephrospheroid, it is not mean to include a kidney or a fragment thereof. It is an ex vivo generated structure in the absence of serum or serum replacement, of about 50 um-500 um.

According to one embodiment the nephrospheroid is capable of generating proximal and distal tubule structures as evidenced by the tubule markers CD13 and AQP1 and the distal tubule markers EMA and SLC12a3.

According to a specific embodiment, the nephrospheroid has an anti-fibrotic activity.

The phrase “non-adherent conditions” or “low adherent conditions” refers to conditions in which the cells do not attach to the surface of a container in which they are cultured such that a substantial portion of the cells can be removed from the surface of the container by mechanical manipulations that do not cause significant damage to the cells. It is understood that the cells can still be retained in or on a non-adherent matrix (e.g., on Hydrogel spheres) and be removed from the surface of the container. Such manipulations include, for example, gentle agitation, massage, or manual manipulation of the container, or rinsing the container with growth media As used herein, a substantial portion of the cells to be removed is at least 70%, preferably at least 75%, 80% or 85%, more preferably at least 90% or 95%. Manipulations that cause damage to the cells can be identified by determining the viability of the cells before and after manipulation, for example by trypan blue staining. Mechanical manipulations should cause damage to less than 20%, preferably less than 15%, or 10%, more preferably less than 5%, 2%, or 1% of the cells. Numerous methods are known for culturing cells under non-adherent conditions. These include growth of cells encapsulated in matrices such as Hydrogel and Matrigel™, on in between layers of agarose, or in Teflon™ bags. An exemplary hydrogel which may be used is PolyHEMA. It will be appreciated that the cells can grow in contact with the non-adherent matrices, but do not adhere to plastic culture containers.

Contemplated culture mediums include, but are not limited to IMDM (Invitrogen) or DMEM (Invitrogen).

According to another embodiment, the culture medium, when generating the nephrospheres is devoid of serum.

The medium may comprise additional components which further encourage the cells to form spheroids. Thus, for example, the medium may further comprise growth factors such as epidermal growth factor (EGF) and fibroblast growth factor (FGF). Other contemplated components include insulin and progesterone.

Thus, specifically, upon reaching 80-100% confluence, cells grown (urine or kidney) as a monolayer are harvested and seeded on low adherence conditions as described above, e.g., pre-coated plates such as with poly (2-hydroxyethylmethacrylate) (poly-HEMA; Sigma-Aldrich), in serum free media, at a concentration of 5.5-13×10⁴ cells/mL. Such a serum-free medium may include a base medium supplemented with antibiotics, amino acids lipids, glucose, transferrin, insulin, growth factors, hormones and minerals.

According to some embodiments of the invention, the medium is termed “SFM” which is composed of N2 medium (Biological Industries) supplemented with 1% Pen-strep 100M, 1% L-glutamine, 0.4% B27 supplement (Gibco), 4 μg/ml heparin sodium (Intramed), 1% non-essential amino acids, 1% sodium pyruvate, 0.2% CD Lipid concentrate (all from Invitrogen), 2.4 mg/ml glucose, 0.4 mg/ml transferrin, 10 mg/ml insulin, 38.66 μg/ml putrescine, 0.04% sodium selenite, 12.6 μg/ml progesterone (all from Sigma-Aldrich), 10 ng/ml FGF and 20 ng/ml EGF.

The cells are grown to spheroids for at least 6 days, e.g., 6-10 days.

Once nephrospheroids are obtained they can be dissociated such as by enzymatic digestion e.g., with TrypLE (GIBCO).

TABLE A
media

| Media | Component/product |
|---|---|
| KSFM:PR | Keratinocyte-SFM Medium |
| | (Kit) with L-glutamine |
| | DMEM-F12 [HAM]1:1 |
| | hydrocortisone |
| | cholera toxin |
| | insulin |
| | transferrin |
| | Animal Free Human EGF |
| RE:MC | REGM Renal Growth |
| | BulletKit |
| | L-glutamine solution |
| | Penicillin-Streptomycin |
| | Solution, *10,000 units/ml |
| | Non-essential Amino acids |
| | (NEAA) |
| | Human PDGF-AB-10 |
| | Animal Free Human EGF |
| | Human FGF-basic |
| | Fbs South American (Ce) |
| | Dulbecco's Modified Eagle |
| | Medium (DMEM) High |
| | Glucose |
| SFM | N2 media |
| | L-glutamine solution |
| | Penicillin-Streptomycin |
| | Solution, *10,000 units/ml |
| | Non-essential Amino acids |
| | (NEAA) |
| | Sodium Pyruvate |
| | Chemically defined (CD) |
| | Lipid mix |
| | B27 supplement (X50) |
| | Heparin sodium Fresenius |
| | 5000 IU/1 ml |
| | Animal Free Human EGF |
| | Human FGF-basic |
| | DMEM/F - 12[ HAM ] 1:1 |
| | Glucose |
| | Transferring |
| | Insulin 10 mg/ml |
| | Putrescine |
| | Sodium Selenite |
| | Progesterone |
| SCM | Iscove's Modified Dulbecco's |
| | Medium (IMDM) with L- |
| | Glutamine |
| | L-glutamine solution |
| | Penicillin-Streptomycin |
| | Solution, *10,000 units/ml |
| | Fbs South American (Ce) |
| | Animal Free Human EGF |
| | Human FGF-basic |
| | Human SCF |

The urine derived epithelial cells (UD-EpC), nephrospheroids (nSPH) and cells derived therefrom can be further qualified for gene expression at the RNA or protein level, e.g., FACS, immunostaining and the like, as shown in the Examples section which follows.

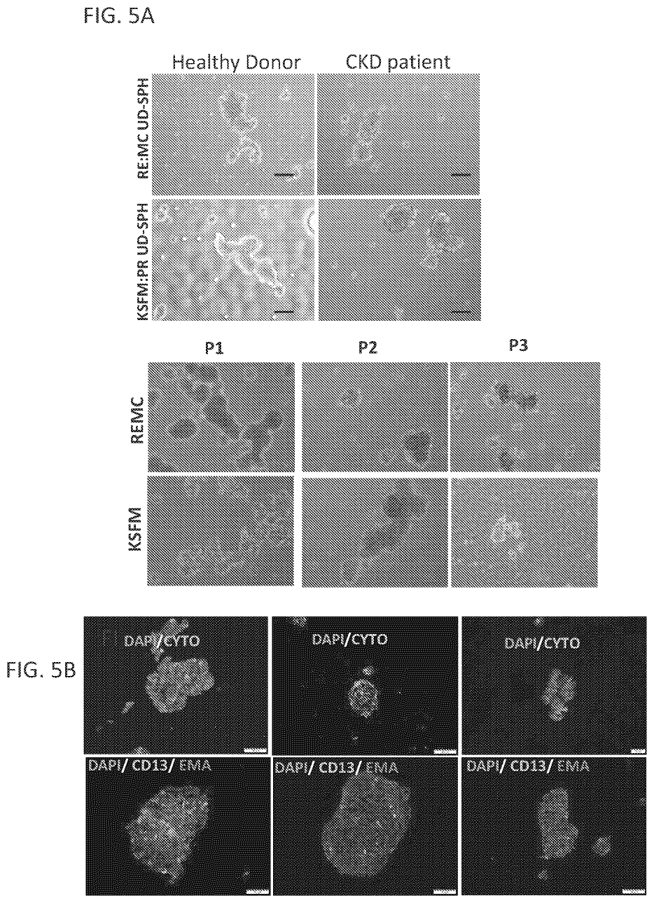

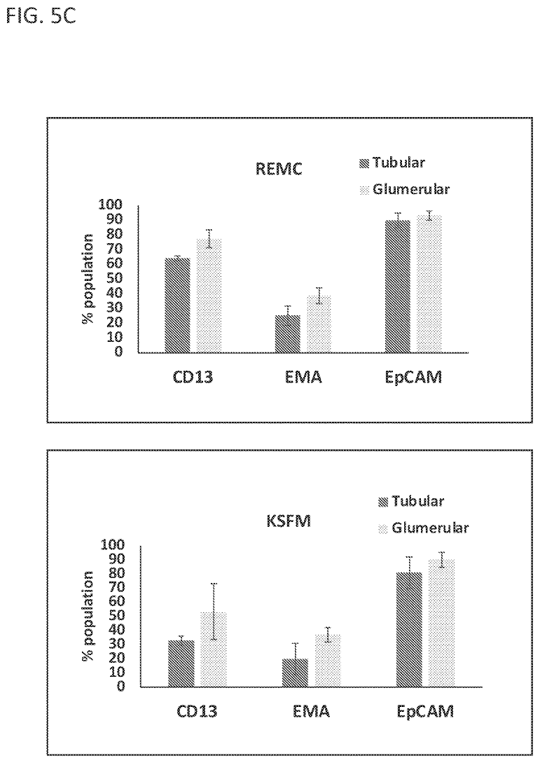

As shown in the Examples section which follows, UD-nSPH express the epithelial marker cytokeratin (as recognized for instance by a pan-keratin wide spectrum screening antibody, DAKO, FIG. 5B) and the kidney segment specific markers CD13 (proximal tubule) and EMA (distal tubule), indicating that they represent genuine nSPH, harboring tubular epithelial cells of different lineages (FIG. 5B). Accordingly, characterization via flow cytometry, for example, demonstrates that the vast majority of cells within UD-nSPH, express the epithelial marker EpCAM, and that both proximal tubular CD13⁺ and, to a lesser extent, distal tubular EMA⁺ cells are present within the nSPH (FIG. 5C). Notably, UD-nSPH derived from CKD patients with tubular and glomerular disorders exhibit similar expression levels of these markers. Taken together, UD-EpC from both healthy individuals and CKD patients are capable of generating nSPH.

According to a specific embodiment, differential gene expression analysis between genes in UD-nSPH and UD-EpC (2D) demonstrates significant inhibition of epithelial-mesenchymal transition (EMT), as evident by down-regulation of SNAI2, SNAI1 and VIM, alongside up-regulation of CDH1, CLDN2 and GRHL2 in UD-nSPH, as determined at the RNA level.

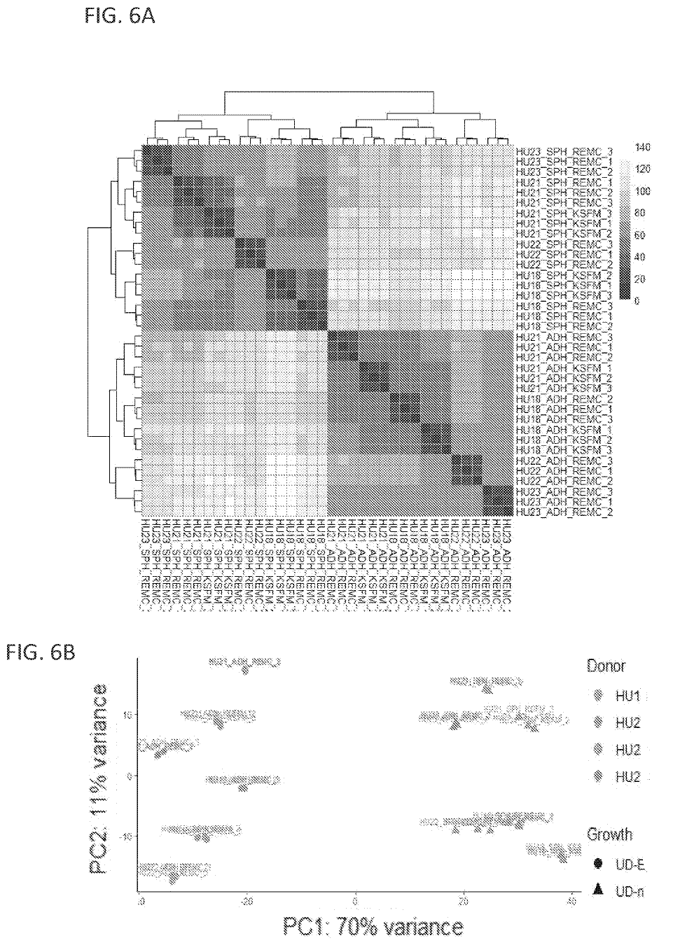

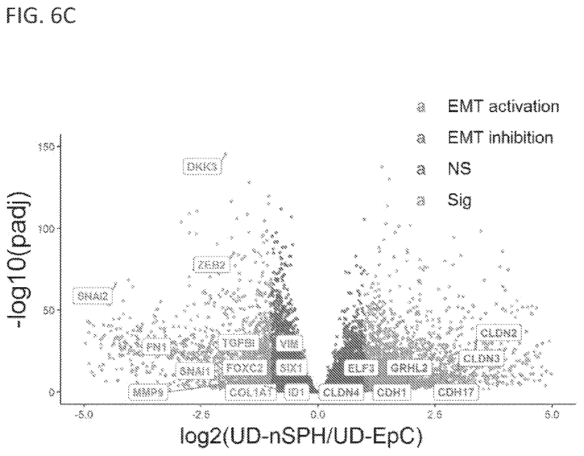

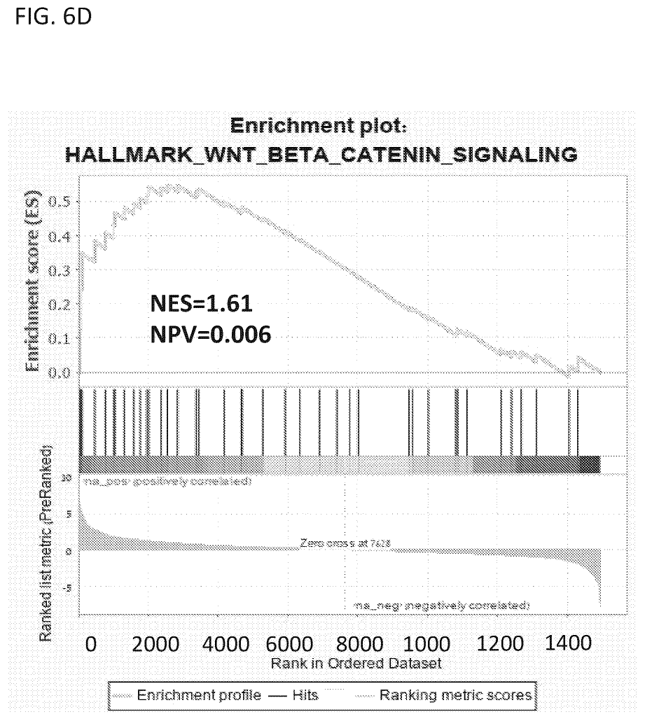

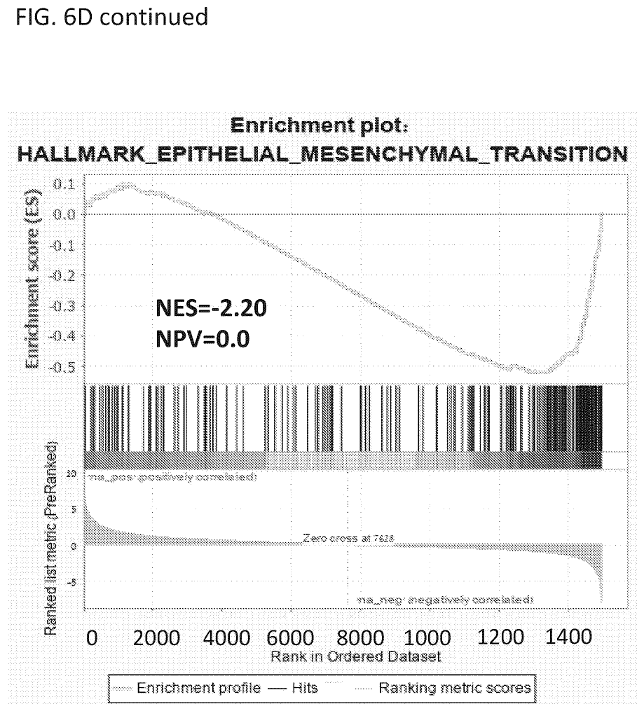

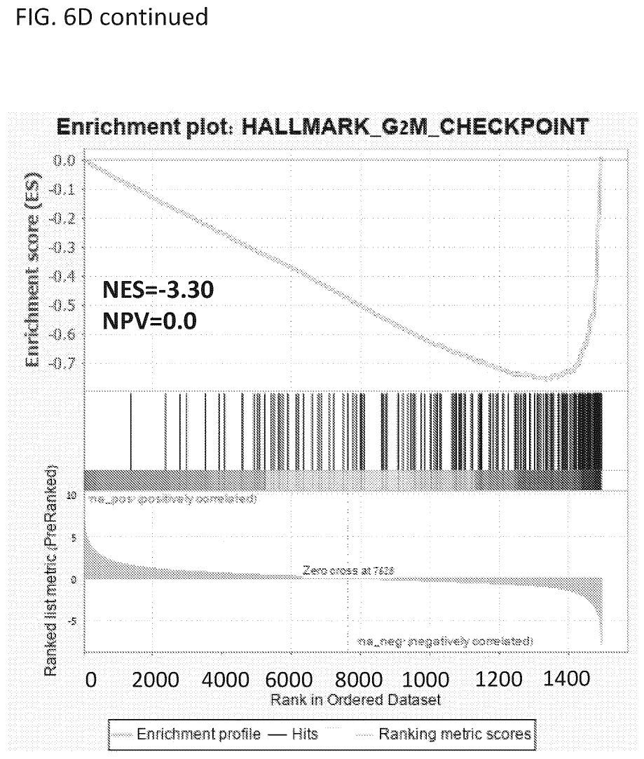

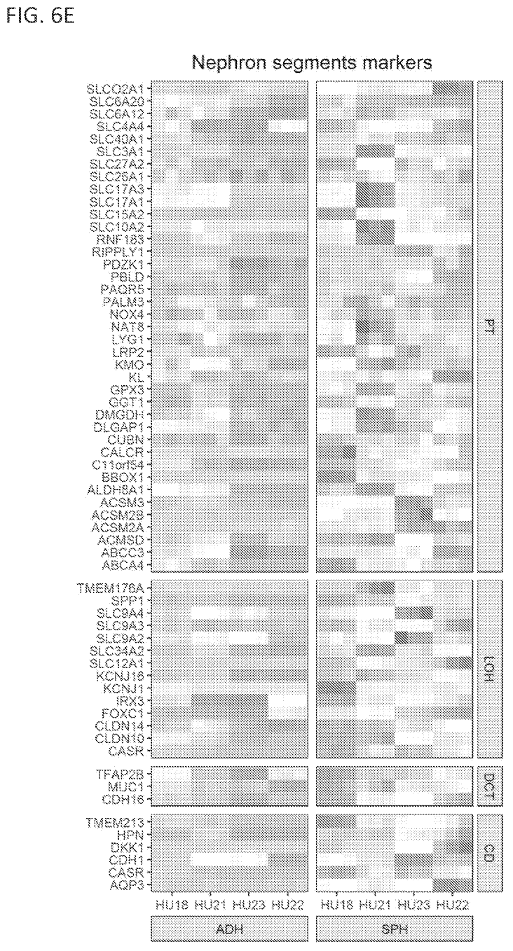

When comparing UD-nSPH to UD-EpC it is found that in the former there is activation of the Wnt signaling pathway and inhibition of cell cycle progression and EMT (FIG. 6D). Consistent with the inhibition of EMT in UD-nSPH, widespread activation of genes encoding for renal tubular epithelial cell types is indicated (FIG. 6E). Taken together, these results demonstrate that UD-nSPH represent renal epithelial structures recapitulating the gene expression of native kidneys.

According to a specific embodiment, the cells (e.g., UD-EpC, nephrospheroids and cells derived therefrom) are non-genetically modified.

According to a specific embodiment, the cells (e.g., UD-EpC, nephrospheroids and cells derived therefrom) can be referred to as primary cells.

According to a specific embodiment, in terms of gene expression the urine derived nephrospheroid exhibits down regulation in gene expression of at least 10%, 20%, 30%, 40%, 50%, 60%, 70%, 80%, 90% or more (out of the differentially expressed (DE) genes) of genes related to cell cycle progression and/or upregulation of nephron specific genes as compared to the 2D cells they are derived from such as when grown in KSFM or REMC.

According to some embodiments, UD-nSPH harbor multipotential tubulogenic capacity in-vivo.

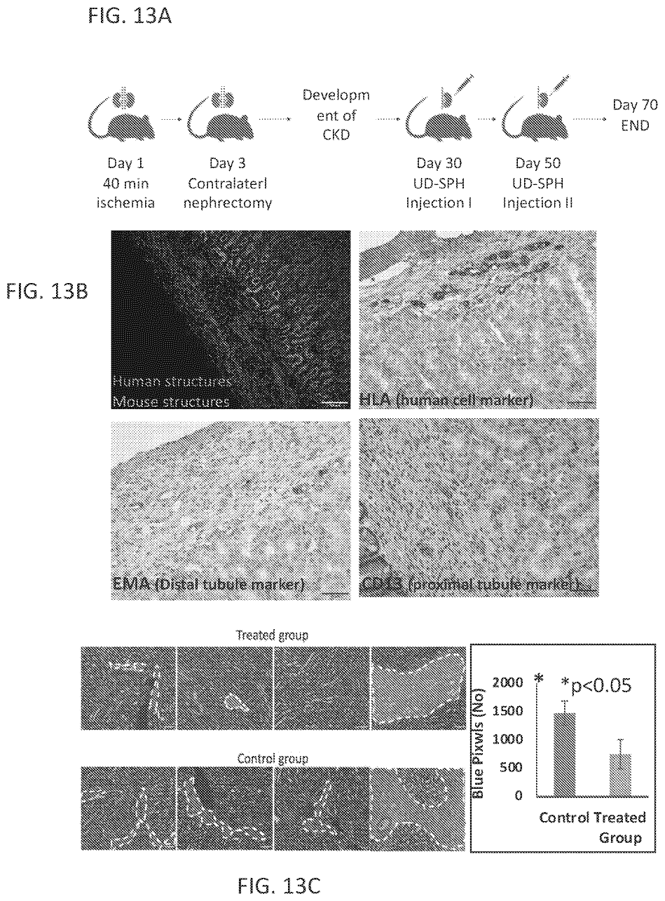

As shown in the Examples section which follows, intra-renal administration of the UD-nSPH shows that tubular structures are formed and stained for the segment specific markers EMA and CD13 (markers of distal and proximal tubules, respectively) indicating renal identity (FIG. 13B). Notably, the fact that the cells have engraftment capacity on top of their tubulogenic potential is highly important from the regenerative perspective, signifying their potential ability to increase the relative proportion of epithelial tissue within fibrotic kidneys without fibrosis.

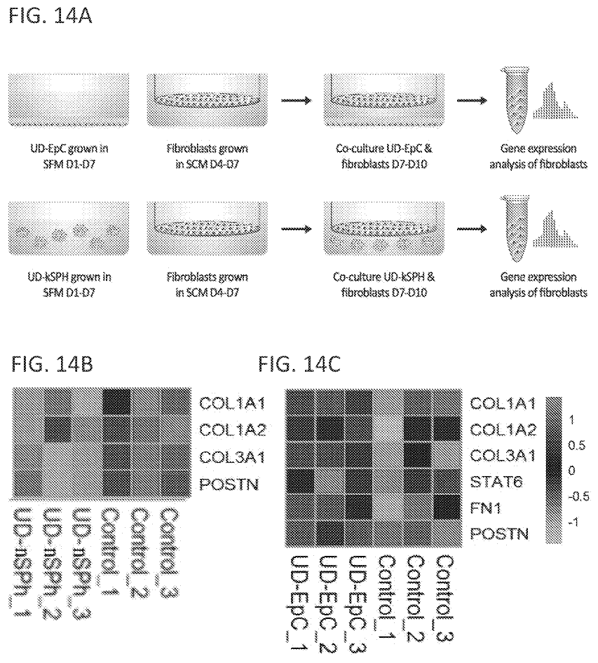

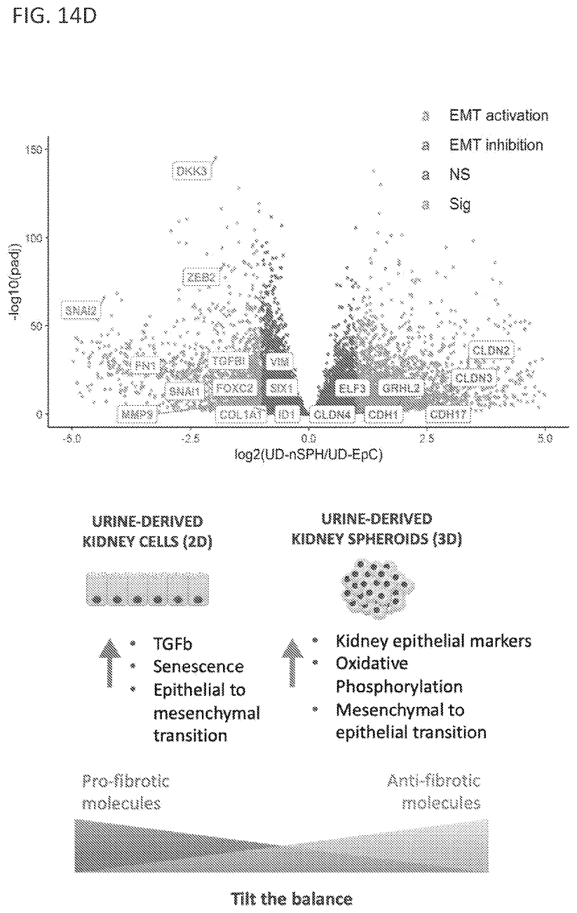

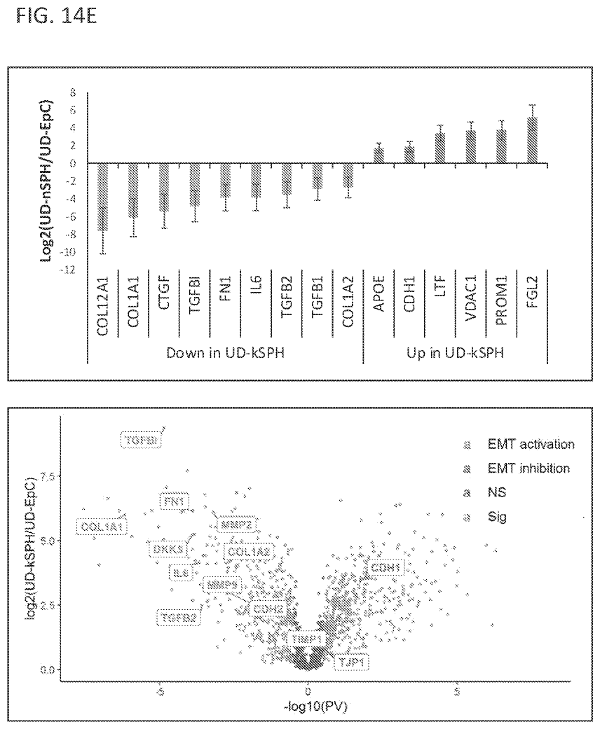

Thus, according to a specific embodiment, the UD-nSPH or secretion thereof (such as in a conditioned medium which includes the secretome) has an anti-fibrotic effect, such as determined by production of fibrosis-related genes, in fibroblasts, including collagen molecules (COL1A1, COL1A2, COL3A1 and PERIOSTIN) indicating a paracrine mechanism of action (FIG. 14B).

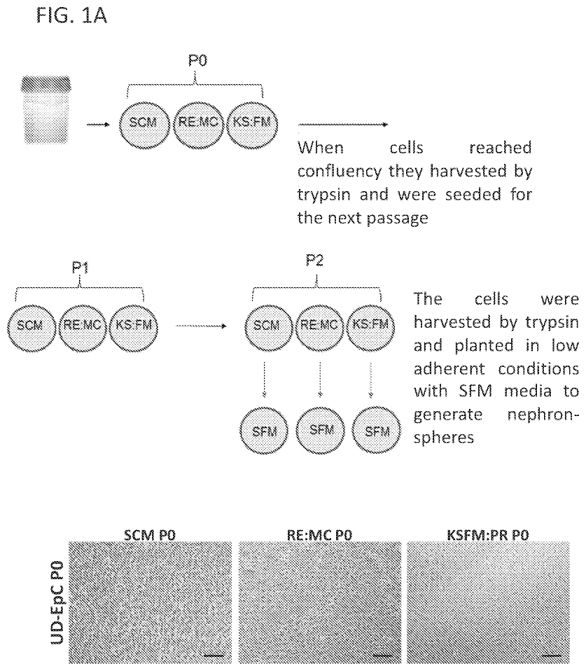

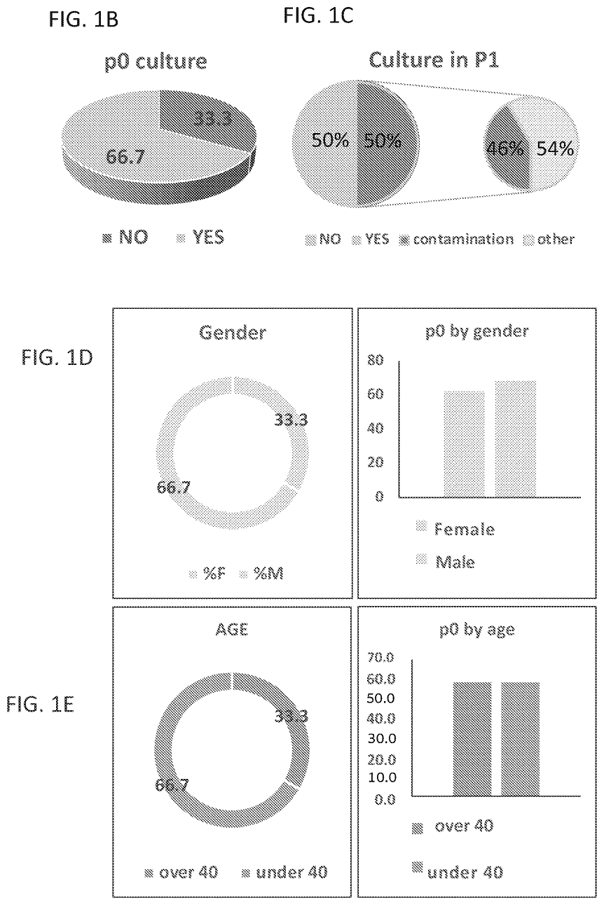

The effect is unique to the UD-nSPH and is not shared by the UD-EpC as further substantiated by the fact that UD-nPSH secretome showed elevation in reno-protective proteins (FGL2 and LTF) and antifibrotic proteins (APOE, CDH1 and VDAC1) (FIG. 14E) along with downregulation in fibrosis related proteins (TGFb, COL1A1, CTGF and FN1) (FIG. 1E), when compared to UD-EpC.

According to a specific embodiment, the nephrospheroid is capable of forming a tubular nephric tissue upon transplantation.

According to a specific embodiment, the nephrospheroid is capable of generating a proximal tubule compartment.

According to a specific embodiment, the proximal tubule compartment expresses Ace2.

According to a specific embodiment, the said nephrospheroid is capable of generating a distal tubule compartment.

According to a specific embodiment, the said nephrospheroid is CD13+/EMA+/EpCAM+/Ace2+ at the protein level and CD13+/EMA+/EpCAM+/Ace2− at the RNA level.

Also provided is a nephrospheroid obtainable according to the method as described herein.

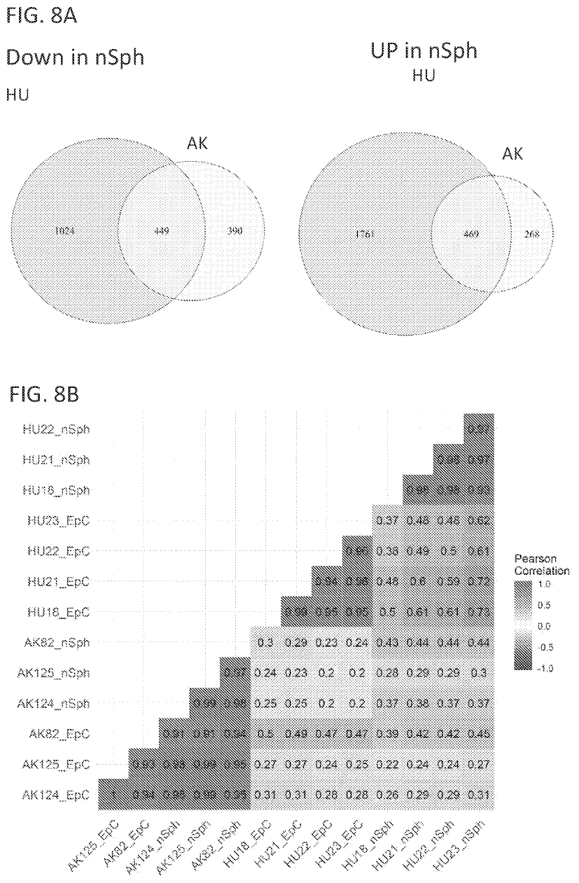

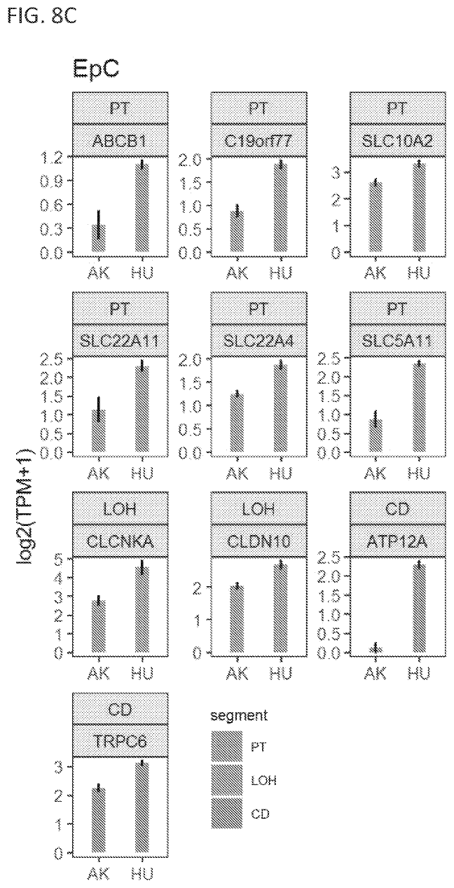

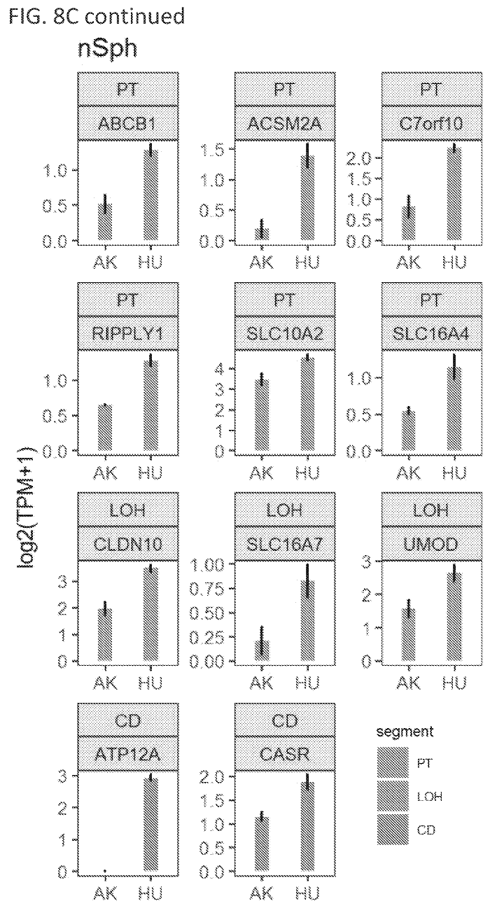

According to a specific embodiment, the nephrospheroid is characterized by gene expression as in FIG. 8C (human urine), e.g., higher expression of ATP12A, ACMS2A and/or SLC16A7 than that derived from human kidney.

According to an aspect of the invention there is provided a nephrospheroid (interchangeably used with neurosphere) comprising urine-derived epithelial cells, the nephrospheroid is capable of forming a tubular nephric tissue upon transplantation.

Also provided are sells or secretome of the nephrospheroid.

As used herein “a secretome” refers to the set of secreted metabolites, proteins expressed by the neurosphere and secreted into the environment, cell-derived particles such as exosomes, nucleic acid molecules (e.g., RNA), carbohydrates, lipids, small molecules which are secreted to the medium in which the nephrospheres are cultured. It can also be referred to as a nephrosoheroid conditioned medium. It may include, for example, cytokines, growth factors, extracellular matrix proteins and regulators, and shed receptors. The secretome can be measured by mass spectrometry as well as other analytic methods.

As shown in the Examples section which follows, the present inventors proved that UD-nSPH harbor multipotential tubulogenic capacity in-vivo, as evidenced by the presence of tubular structures that were formed following implantation and stained for the segment specific markers EMA and CD13 (markers of distal and proximal tubules, respectively) indicating renal identity. UD-nSPH actively inhibited the production of fibrosis-related genes, in fibroblasts, including collagen molecules (COL1A1, COL1A2, COL3A1 and PERIOSTIN) indicating a paracrine mechanism of action.

Thus, according to an aspect of the invention there is provided a method of regenerating renal function, the method comprising administering to a subject in need thereof a therapeutically effective amount of the nephrospheroids or cells or secretome, thereby regenerating renal function.

Thus, nephrospheroids or cells or secretome of the present invention can be used to treat any form of acute or chronic kidney disease, diabetic nephropathy, renal disease associated with hypertension, hypertensive acute tubular injury (ischemic, toxic), interstitial nephritis, congenital anomalies (Aplasia/dysplasia/obstructive uropathy/reflux nephropathy); hereditary conditions (Juvenile nephronophtisis, ARPCKD, Alport, Cystinosis, Primary Hyperoxaluria); Glomerulonephritides (Focal Segmental Glomerulosclerosis); Multisystem Diseases (SLE, HSP, HUS) or any form of genetic or inherited glomerular, tubular, tubulo-interstitial kidney disease.

The present teachings contemplate administration of single cell suspensions of dissociated spheroid-cells, partly dissociated spheroid-cells or non-dissociated spheroid cells. Each of these should be considered as a separate independent embodiment of the invention.

The cells in any form or secretome may be administered per se or as part of a pharmaceutical composition where they are mixed with a suitable carrier or excipient.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the cells in any form or secretome accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

The cells in any form or secretome can be administered into a subject such as surgically or by infusion. For example, renal cells are injected in vivo into a kidney that is in the postischemic recovery phase. This can be tested easily in an animal model predictive of ischemic kidney damage, the renal pedicle of an anesthetized mouse is clamped for 30 minutes to induce kidney ischemia. Renal stem cells are then injected into the juxtamedullary region (approximately 2000 cells at a depth of 2-4 mm). Otherwise, the cell secretome is injected into the renal artery/tail vain. After 2 weeks of recovery, immunohistochemical analysis is used as described above to look for differentiated cells surface markers GP330, Tamm-Horfall, Dolichos Biflorous, and the like. In addition, immunohistochemical analysis for collal and chemical staining (such as: Masson-Trichrome and Sirius red) are used to quantify the fibrotic tissue. Post-incorporation differentiation status can then be compared to pre-injection marker status.

The examples section which follows, show intra-renal administration of the nephrospheres, which is contemplated herein.

The cells in any form or secretome can be used to construct artificial kidney systems. Such a system can be based on a hollow fiber filtration system.

In one example of a filtration device, the nephrospheres or cells derived therefrom are grown on the interior of hollow fibers having relatively high hydraulic conductivity (i.e., ultrafiltration coefficient). The hollow fiber passes through a chamber that is provided with a filtrate outlet port. Arterial blood containing metabolic waste and other unwanted material is introduced into one end of the hollow fiber through an inlet port. Blood passed through the fiber and exits the other end of the fiber through an outlet port where it passed into the patient's vascular venous flow. As blood passes through the fiber, filtrate pass through the nephrospheres or cells derived therefrom lining the interior of the fiber and through the hollow fiber itself. This filtrate then passes out of the chamber containing the fiber through the filtrate outlet port. The device preferably includes many such hollow fibers each of which can be in its own chamber. Alternatively many, many hollow fibers (100-100,000 or even more) can be bundled together in a single chamber.

The nephrospheres or cells derived therefrom of the invention can be used to create a tubule-processing device. In such a device the nephrospheres or cells derived therefrom can be grown in a layer on the exterior of the semipermeable hollow fiber (i.e. a scaffold). The fiber is placed in a chamber that is provided with an inlet port and an outlet port. As ultrafiltrate from filtered blood flows through the chamber, reabsorbant passes through the cell layer and through the wall of the fiber into the lumen of the fiber from which it can be directed back into the patient's systemic circulation. Ultrafiltrate that is not reabsorbed passes through the outlet port of the chamber.

In the devices described above, it can be desirable to coat the fiber surface that will bear the cell layer with extracellular matrix components. For example, the fiber can be coated with materials such as collagen (e.g., Type I collagen or Type IV collagen), proteoglycan, fibronectin, and laminin or combinations thereof. It can be desirable to combine various cell types on the inner or outer surface of the fibers. For example, it can be desirable to include endothelial cells and pericyte, vascular smooth muscle cells or mesangial cells or fibroblasts or combinations thereof. It can also be useful to provide a feeder layer of cells, e.g., irradiated fibroblasts or other cells that can provide soluble factors and structural support to cells they are indirectly or directly in contact with.

The above-described filtration system and the above-described tubule processing system can be combined to create an artificial kidney. Such systems are described in U.S. Pat. No. 6,150,164, hereby incorporated by reference. A number of suitable materials for forming the hollow fiber are described in U.S. Pat. No. 6,150,164, hereby incorporated by reference.

The present invention provides a method of using the cell populations of the present invention to characterize cellular responses to biologic or pharmacologic agents involving producing the cells as described herein and culturing with one or more biologic or pharmacologic agents, identifying one or more cellular responses to the one or more biologic or pharmacologic agents, and comparing the one or more cellular responses of the cultures. Tissue culture techniques known to those of skill in the art allow mass culture of hundreds of thousands of cell samples from different individuals, providing an opportunity to perform rapid screening of compounds suspected to be, for example, teratogenic or mutagenic.

The expression of Ace2 on the membrane of the nephrospheres or cells derived therefrom makes them a good model for Coronavirus infection. According to a specific embodiment the kidney EpC and UD-EpC as well as spheres derived therefrom express Ace2. This finding substantiate their use as a model for a Coronavirus infection such as SARS-CoV-02 or variants thereof, as further described hereinbelow. The generation of kidneyspheroids have been described in US 2013-0059325, which is hereby incorporated by reference in its entirety Thus, according to an aspect of the invention, there is provided a method of drug design, the method comprising determining an effect of a test drug on the nephrospheroid or cells or secretome as described herein.

According to a specific embodiment, the drug is a small molecule, carbohydrate, proteinaceous, nucleic acid or combination of same.

When determining the efficacy for treating or preventing infection of a virus, the determining step is performed in the presence of the infective agent, e.g., virus, e.g., Coronavirus, according to some embodiments.

As used herein, “Coronavirus” refers to enveloped positive-stranded RNA viruses that belong to the family Coronaviridae and the order Nidovirales.

Examples of Corona viruses which are contemplated herein include, but are not limited to, 229E, NL63, OC43, and HKU1 with the first two classified as antigenic group 1 and the latter two belonging to group 2, typically leading to an upper respiratory tract infection manifested by common cold symptoms.

However, Coronaviruses, which are zoonotic in origin, can evolve into a strain that can infect human beings leading to fatal illness. Thus particular examples of Coronaviruses contemplated herein are SARS-CoV, Middle East respiratory syndrome Coronavirus (MERS-CoV), and the recently identified SARS-CoV-2 [causing 2019-nCoV (also referred to as “COVID-19”)].

It would be appreciated that any Corona virus strain is contemplated herein even though SARS-CoV-2 is emphasized in a detailed manner.

According to specific embodiments, the Corona virus is SARS-CoV-2.

As used herein the SARS-CoV-2 includes any variants and mutants thereof including, but not limited to, the B.1.1.7 (Alpha), B.1.351 (Beta), B.1.617.2 (Delta), P.1 (Gamma), B.1.526 (Iota), B.1.427 (Epsilon), B.1.429 (Epsilon), B.1.617 (Kappa, Delta), B.1.525 (Eta) and P.2 (Zeta). The present inventors have synthesized proteins of some of these variants, referred herein as “variants of concern” or “VOCs” to support the use of the antibodies or combinations thereof of some embodiments of the invention is combating wild type viruses and variants thereof.

These teachings can be further harnessed towards tailored therapy against a disease.

Thus, according to an aspect of the invention, there is provided a method of personalized therapy, the method comprising:
(a) contacting a Coronavirus-infected renal epithelial cell culture or nephrospheroid produced of said culture with a test drug; and
(b) determining an alleviation in viral load following said contacting, said alleviation being indicative of an efficacious therapy.

According to a specific embodiment, the said renal culture or nephrospheroid is autologous.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Ethics Statement

This study was conducted according to the principles expressed in the Declaration of Helsinki. The study was approved by the Institutional Review Boards of Sheba Medical Center. Human kidney samples were retrieved from borders of renal cell carcinoma (RCC) tumors from partial and total nephrectomy patients, from both Sheba Medical Center and Wolfson hospital. This procedure was done after obtaining informed consent and has been approved by the local ethical committee.

All animal experiments were approved by the Committee for Ethics in Animal experimentation of the Sheba Medical Center.

Establishment of Primary Culture from Urine Samples

Mid-stream urine samples were collected from 24 healthy individuals and 12 CKD patients who provided their informed consent and following the approval of The Chaim Sheba Medical Center ethics committee. The urine samples were centrifuged at 400×g, washed with PBS and centrifuged again at 200×g. Then the cell pellet was seeded on three different gelatin-coated wells (0.1%, Biological Industries, 01-944-1B), containing one of the following media: 1. IMDM-SCM (serum-containing medium); 2. KSFM:PR (keratinocyte serum free medium: progenitor cell medium at a 1:1 ratio); 3. RE:MC (renal growth medium: Mesenchymal Cell proliferation at a 1:1 ratio). Thereby establishing P0, (Table 1 media). When cells reached confluency they were harvested by trypsin and seeded to the next passage.

In order to increase cell proliferation the present inventors added into the growth media NRG1 or CD40L, in different concentrations: 2, 5 and 10 ng/ml, each one separately.

Establishment of Primary Culture from Human Kidney

Human kidney samples were retrieved from borders of renal cell carcinoma (RCC) tumors from partial and total nephrectomy patients. Collected tissues were washed with PBS, weighed and minced into ˜1 mm pieces, using sterile surgical scalpels. The dissected tissue was then incubated for two hours in 37° C. with Iscoves' Mod Dulbecco's Medium (IMDM) (Invitrogen) supplemented with 0.1% collagenase IV (Invitrogen). The digested tissue was then forced through 100 pm cell strainers to achieve a single cell suspension. After the digesting medium was removed, the cells were resuspended in a growth medium and plated on T75 flasks. Serum containing medium (SCM) was comprised of IMDM (Biological Industries) supplemented with 10% fetal bovine serum (Invitrogen), 1% Pen-strep 100M, 1% L-glutamine (both from Biological industries), 100 ng/ml EGF, 100 ng/ml bFGF and 10 ng/ml SCF (all growth factors purchased from Peprotech Asia). Cells were detached using 0.05% Trypsin/EDTA (Invitrogen) when reached confluency and cryopreserved in 10% DMSO FBS. Two dimensional (2D) KD-EpC were photographed using Nikon Eclipse TS100 and Nikon Digital Sight cameras.

nSPH Formation and Dissociation

Upon reaching 80-100% confluence, cells grown (urine or kidney) as a monolayer were harvested and seeded on poly (2-hydroxyethylmethacrylate) (poly-HEMA; Sigma-Aldrich) pre-coated plates, in serum free media (SFM), at a concentration of 5.5-13×10⁴ cells/mL. SFM was comprised of N2 medium (Biological Industries) supplemented with 1% Pen-strep 100M, 1% L-glutamine, 0.4% B27 supplement (Gibco), 4 μg/ml heparin sodium (Intramed), 1% non-essential amino acids, 1% sodium pyruvate, 0.2% CD Lipid concentrate (all from Invitrogen), 2.4 mg/ml glucose, 0.4 mg/ml transferrin, 10 mg/ml insulin, 38.66 μg/ml putrescine, 0.04% sodium selenite, 12.6 μg/ml progesterone (all from Sigma-Aldrich), 10 ng/ml FGF and 20 ng/ml EGF. nSPH were photographed using Nikon Eclipse TS100 and Nikon Digital Sight cameras. In order to dissociate the nSPH into single cells, nSPH were collected and incubated with TrypLE (GIBCO) For 10 min. nSPH-derived cells were then counted for further test

TABLE 1
media

| Media | Component / product | Cut. No. | Manufacturer |
|---|---|---|---|
| KSFM:PR | Keratinocyte-SFM Medium | 17005075 | ThermoFisher |
| | (Kit) with L-glutamine | | (Gibco) |
| | DMEM-F12 [HAM]1:1 | 01-055-1A | Biological |
| | | | Industries |
| | hydrocortisone | H0888-1G | SIGMA |
| | cholera toxin | C8052 | SIGMA |
| | insulin | I9278-5ML | SIGMA |
| | transferrin | T8158 | SIGMA |
| | Animal Free Human EGF | AF-100-15-1000 | Peprotech |
| RE:MC | REGM Renal Growth | CC-3190 | Lonza |
| | BulletKit | | |
| | L-glutamine solution | 03-020-1A | Biological |
| | | | Industries |
| | Penicillin-Streptomycin | 03-031-1B | Biological |
| | Solution, *10,000 units/ml | | Industries |
| | Non-essential Amino acids | 11140-035 | ThermoFisher |
| | (NEAA) | | (Gibco) |
| | Human PDGF-AB-10 | 100-00AB-10 | Peprotech |
| | Animal Free Human EGF | AF-100-15-1000 | Peprotech |
| | Human FGF-basic | 100-18B-1000 | Peprotech |
| | Fbs South American (Ce) | 10270106 | ThermoFisher |
| | | | (Gibco) |
| | Dulbecco's Modified Eagle | 01-055-1A | Biological |
| | Medium (DMEM) High | | Industries |
| | Glucose | | |
| SFM | N2 media | 01-058-1A | Biological |
| | | | Industries |
| | L-glutamine solution | 03-020-1A | Biological |
| | | | Industries |
| | Penicillin-Streptomycin | 03-031-1B | Biological |
| | Solution, *10,000 units/ml | | Industries |
| | Non-essential Amino acids | 11140-035 | ThermoFisher |
| | (NEAA) | | (Gibco) |
| | Sodium Pyruvate | 11360-039 | ThermoFisher |
| | | | (Gibco) |
| | Chemically defined (CD) | 11905-031 | ThermoFisher |
| | Lipid mix | | (Gibco) |
| | B27 supplement (X50) | 17504-044 | ThermoFisher |
| | | | (Gibco) |
| | Heparin sodium Fresenius | | |
| | 5000 IU/1 ml | | |
| | Animal Free Human EGF | AF-100-15-1000 | Peprotech |
| | Human FGF-basic | 100-18B-1000 | Peprotech |
| | DMEM/F-12[ HAM ] 1:1 | 01-170-1A | Biological |
| | | | Industries |
| | Glucose | (G7021 | SIGMA |
| | Transferring | T8158 | SIGMA |
| | Insulin 10 mg/ml | I9278 | SIGMA |
| | Putrescine | P5780 | SIGMA |
| | Sodium Selenite | S9133 | SIGMA |
| | Progesterone | P8783 | SIGMA |
| SCM | Iscove's Modified | 01-058-1A | Biological |
| | Dulbecco's Medium | | Industries |
| | (IMDM) with L-Glutamine | | |
| | L-glutamine solution | 03-020-1A | Biological |
| | | | Industries |
| | Penicillin-Streptomycin | 03-031-1B | Biological |
| | Solution, *10,000 units/ml | | Industries |
| | Fbs South American (Ce) | 10270106 | ThermoFisher |
| | | | (Gibco) |
| | Animal Free Human EGF | AF-100-15-1000 | Peprotech |
| | Human FGF-basic | 100-18B-1000 | Peprotech |
| | Human SCF | 300-07-100 | Peprotech |

Immunofluorescence Staining (IF)

5 mM Paraffin sections were pre-treated using OmniPrep solution (pH 9.0) at 95° C. for one hour in accordance with the manufacturer's protocol (Zytomed Systems). Blocking was done using Cas-Block solution (Invitrogen immunodetection, 00-8120) for one hour followed by one hour incubation with the following primary antibodies: anti-HLA (Rb; 1/200; abcam, 52922), anti-EMA (Ms; 1/2 of the prediluted antibody; Cell Marque, 247M-98), anti-CD13 (Rb; 1/400; abcam, 108382) AQP1 (abcam 168387) and SLC12A3 (Merck AB3553). Detection was done using alexa488 conjugated anti-rabbit and alexa555 conjugated anti-mouse secondary antibodies (Invitrogen) for 60 min. DAPI-containing mounting (Dapi Fluoromount-G; SouthernBiotech, 0100-20) was applied. Slides were analyzed using Olympus BX51 fluorescence microscope and Olympus DP72 camera.

FACS

0.5×10⁵ cells were suspended in FACS buffer (0.5% BSA in 1×PBS). The cells were then incubated with a primary antibody or an isotype control (Table 2). Cell viability was tested using 7AAD viability staining solution (eBioscience). Cell labeling was detected using FACSCalibur (BD Pharmingen). Results were analyzed using FlowJo analysis software.

TABLE 2
FACS antibodies

| antibody | catalog no. | Isotype | catalog no. | manufacturer |
|---|---|---|---|---|
| Anti-Human | NB120- | Mouse IgG1 K | 53-4714-42 | eBioscience |
| CD227 | 22711AF488 | Isotype control | | |
| (Mucin 1) | | | | |
| Alexa 488 | | Alexa 488 | | |
| Anti-Human | 12-9326-42 | Mouse IgG1 k-PE, | 12-4714-81 | eBioscience |
| CD326 | | Isotype control | | |
| (EpCAM) | | | | |
| PE | | | | |
| CD13-APC | 17-0138-42 | Mouse IgG1 | 17-4714-81 | eBioscience |
| | | k-APC, | | |
| | | Isotype control | | |

Grafting Cells in NOD/SCID Mice (Tubule Formation Assay)

2×10⁶ nSPH-derived cells were suspended in 200 μL Matrigel (MG) (BD Biosciences) and were injected subcutaneously into NOD/SCID mice (Harlan Laboratories, Israel). 14 days following injection, the grafts were removed, paraffin-embedded, and sectioned for IF analyses.

Fibrosis Assay

Co-culturing of UD-EpC with fibroblasts: UD-EpC were grown separately in RE:MC for 7 days. Fibroblasts were grown separately in SCM starting on day 4 on ThinCerts™ inserts (Greiner bio-one) for three days. On day 7 the inserts with the fibroblasts of the test group were transferred to 6 wells plate in co-culture above wells with UD-EpC-2D. On day 7 in the control group, the inserts with the fibroblasts were transferred to 6 wells plate in co-culture above wells with REMC without UD-EpC-2D.

Co-culturing of UD-SPH with fibroblasts: UD-SPH were grown separately in SFM for 7 days in polyhema-coated 6 well plates. Fibroblasts were grown separately in SCM on ThinCerts™ inserts (Greiner bio-one) for three days starting on day 4. On day 7 the inserts with the fibroblasts of the test group transferred to wells of 6 wells plate in co-culture with UD-SPH. In the control group on day 7 the inserts with the fibroblasts transferred to wells of 6 wells well with SFM without UD-SPH.

On day 10, all the above fibroblasts were harvested for RNA purification. Total RNA was prepared using Direct-zol RNA MiniPrep kit (Zymo Research) according to the manufacturer's instructions. Gene expression analysis was carried out for 4 fibrosis related genes: COL1A1, COL2A1, PERIOSTIN, COL3A, STAT6 and FN.

Mouse Model of Chronic Disease in NOD-SCID Mice

10 NOD-SCID mice underwent reperfusion protocol described in FIG. 23 a. On day 1 all mice underwent 40 minutes of ischemia by clumping their renal artery of one of their kidneys. On day 3 all mice underwent contralateral nephrectomy (non-ischemic kidney) in order to prevent spontaneous recovery. On day 30 and 51 the treatment group was injected directly to the kidney parenchyma with UD-SPH cells. Spheroids underwent gentle mechanical dismantling before injection. The control group underwent the same procedure without cells.

Masson-Trichrome Staining for Fibrosis

Staining was done according to (Kramann & al 2015)⁵⁸. After staining 30 slices of paraffin embedded mouse kidney tissue of each group (treated and untreated), quantification of the blue pixels was carried out by photoshop.

### Example 1

Human Epithelial Cell Cultures Can Be Established from Urine Samples of Healthy Individuals In order to establish a primary culture of epithelial cells from urine samples, several protocols were tested, using urine derived from healthy individuals. A total of 24 urine samples from 24 healthy individuals (Table 3) were collected. The cells from each sample were equally divided into three separate gelatin-coated wells, each containing a different growth medium, thereby establishing passage 0 (P0) culture (FIG. 1A). The three types of media used were: 1. IMDM-SCM (serum-containing medium); 2. RE:MC (renal growth medium: MC proliferation kit at a 1:1 ratio); 3. KSFM:PR (keratinocyte serum free medium: progenitor cell medium at a 1:1 ratio). (Table 1). The following day, a monolayer of epithelial cells, which was termed ‘urine-derived epithelial cells’ (UD-EpC), could be detected in all media types. Within 14-21 days, the cultures reached confluence, at which point they were sub-cultured into P1, maintaining the same type of medium for each group. Following 4-12 days, the cells were again sub-cultured into P2, in which they were allowed to grow for 7 days before transferring them into low adherence culture conditions, thereby generating nSPH s (FIG. 1A). Approximately 70% of urine samples successfully generated a P0 epithelial culture. Of the latter, approximately 50% successfully generated a P1 culture. The leading reason for the low rate at this stage to grow in P1 (˜46%), was bacterial contamination, likely arising from improper collection of urine by the donor (FIGS. 1B and 1C). No significant difference in culturing success rates was detected between men and women, which represented approximately 67% and 33% of donors, respectively. Similarly, donor age did not affect the ability to generate a cell culture, as demonstrated when comparing donors above and below 40 years of age (˜67% and 33% of donors, respectively) (FIGS. 1D and 1E).

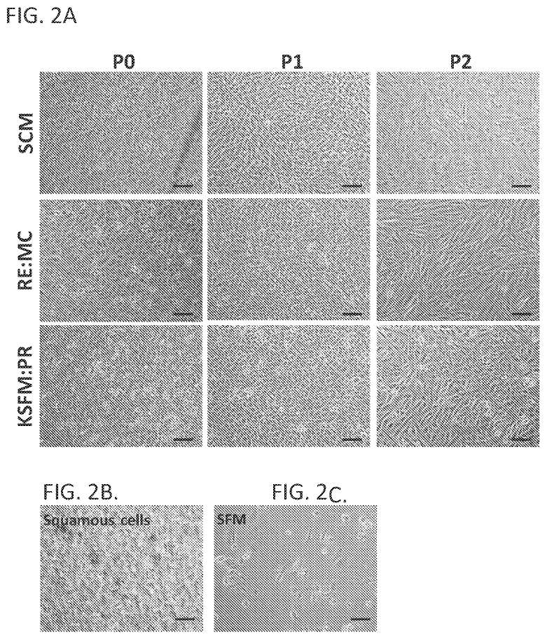

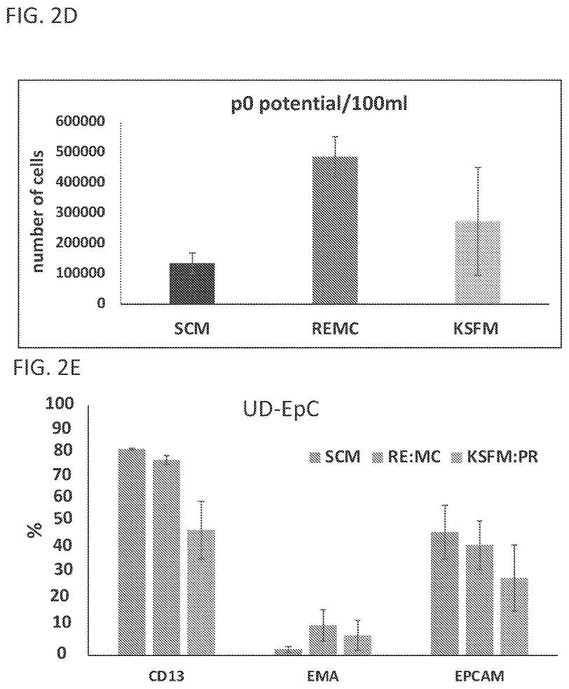

When comparing the three types of media, UD-EpC exhibited different morphology and growth characteristics in each of the media. Growth in SCM resulted in relatively slower proliferation and a more elongated morphology, whereas cells grown in the other media types demonstrated faster proliferation and a more cuboidal phenotype (FIG. 2A). Notably, initial urine samples consisted, aside from epithelial cells, also of squamous cells (mostly observed in samples derived from women, likely of urethral origin) (FIG. 2B), leukocytes and semen, in accordance with previous reports²⁵. These cell types did not adhere to the plate surface and gradually disappeared following a few medium aspirations. Interestingly, urine samples cultured in SFM did not proliferate, failing to reach P1 (FIG. 2C), indicating the necessity of serum for UD-EpC growth in-vitro. While UD-EpC successfully expanded in all three media types, significant variability in growth kinetics was seen, as noted above. Cells grown in REMC exhibited the highest growth potential, generating an average of 2.6×10⁵ P0 cells per 100 ml of urine. Next, KSFM:PR-grown cells reached an average of 1.7×10⁵ cells/100 ml. Lastly, cells cultured in SCM generated 0.5×10⁵ cells/100 ml (FIG. 2D). To further delineate the identity of UD-EpC, the expression of the epithelial marker EpCAM, the proximal tubule marker CD13 and distal tubule marker EMA in UD-EpC grown was determined in each of the media types, via flow cytometry (The vast majority of cells were EpCAM+, consistent with their epithelial phenotype (FIG. 2E). All cultures consisted of both proximal and distal tubular cells, with a predominance of the former (FIG. 2E). Taken together, these results demonstrate that primary cultures of human UD-EpC can be generated by a simple and cost-effective protocol, and that REMC and KSFM are the optimal media for establishing UD-EpC cultures.

TABLE 3

| | | | | | | | |
|---|---|---|---|---|---|---|---|
| Patient | Gender | Age | Weight | CKD | | | Additional |
| ID | (M/F) | (Years) | (Kg) | stage | Therapies | Background | information |

Healthy Controls

| | | | | | | |
|---|---|---|---|---|---|---|
| HU1 | M | 36 | N/A | N/A | None | |
| HU2 | F | 45 | N/A | N/A | None | |
| HU3 | F | 31 | N/A | N/A | None | |
| HU4 | M | 31 | N/A | N/A | None | |
| HU5 | M | 43 | N/A | N/A | None | |
| HU6 | F | 34 | N/A | N/A | None | |
| HU7 | M | 73 | N/A | N/A | None | |
| HU8 | M | 35 | N/A | N/A | None | |
| HU9 | M | 34 | N/A | N/A | None | |
| HU10 | M | 47 | N/A | N/A | None | |
| HU11 | M | 45 | N/A | N/A | None | |
| HU12 | M | 50 | N/A | N/A | None | |
| HU13 | M | 29 | N/A | N/A | None | |
| HU14 | F | 40 | N/A | N/A | Levothyroxine | Hypothyroidism |
| HU15 | M | 41 | N/A | N/A | None | |
| HU16 | F | 37 | N/A | N/A | None | |
| HU17 | M | 36 | N/A | N/A | None | |
| HU18 | F | 35 | N/A | N/A | None | |
| HU26 | M | 29 | N/A | N/A | None | |
| HU30 | M | 40 | N/A | N/A | None | |
| HU31 | F | 34 | N/A | N/A | None | |
| HU33 | M | 35 | N/A | N/A | None | |
| HU34 | M | 37 | N/A | N/A | None | |
| HU36 | M | 35 | N/A | N/A | None | |

### Example 2

RE:MC and KSFM:PR are the Optimal Media for UD-EpC expansion Having shown that UD-EpC can be established from urine samples of healthy individuals, the ability to generate similar cultures from CKD patients was next tested. For this purpose, urine samples were collected from 12 CKD patients (Table 4), representing both glomerular and tubular disorders. Surprisingly, the same protocol enabled the establishment of primary cultures of UD-EpC, exhibiting similar morphology to UD-EpC derived from healthy individuals (FIG. 3A). Notably, when cultured in RE:MC, UD-EpC derived from CKD patients exhibited a significantly higher cell yield, compared to KSFM (FIG. 3B). Flow cytometric analysis comparing UD-EpC from patients with tubular vs. glomerular diseases, revealed that growing the cells in RE:MC resulted in a mostly epithelial phenotype of the cells, as evident by EpCAM expression (FIG. 3C). In contrast, culturing the cells in KSFM resulted in a significantly lower percentage of epithelial cells, especially in the case of tubular diseases when considering the expression levels of the epithelial marker EpCAM. In both media types, the majority of cells were of the proximal phenotype, as evident by the expression of CD13 and EMA (FIG. 3C). Taken together, these results indicate that UD-EpC can be generated from urine samples of CKD patients, regardless of the underlying etiology, and that REMC is the optimal medium in preserving the epithelial phenotype of the cells.

The present inventors carried out global gene expression analysis of UD-nSPH by comparing them to the 2D UD-EpC from which they were derived, via RNA-sequencing, using 2D cultures and nSPH obtained from one healthy donor and three CKD patients, each grown in either REMC or KSFM. First the present inventors compared UD-EpC that were cultured in REMC to those cultured in KSFM. Gene set enrichment analysis (GSEA) showed that there was down-regulation of genes related to cell cycle progression in cells that were cultured with KSFM (FIG. 3D). Cells grown in REMC exhibited the highest growth potential, generating an average of 2.6×105 P0 cells per 100 ml of urine, while KSFM-grown cells reached an average of 1.7×105 cells/100 ml). In addition, it was shown that in comparison to REMC-grown cells, there was down-regulation of nephron segment specific markers in KSFM-grown cells (FIG. 3E).

Generally, nephrospheroids are less proliferative hence the cell cycle genes are downregulated. When comparing 2D cells that were grown in two different media: REMC and KSFM, and it can be seen that the cells that grew with KSFM express lower levels of cell cycle genes and kidney specific genes, these results indicated that REMC is a better choice for growing adherent kidney cells isolated from urine.

TABLE 4

| | | | | | | | | |
|---|---|---|---|---|---|---|---|---|
| Patient | Gender | Age | Weight | Kidney | CKD | | | Additional |
| ID | (M/F) | (Years) | (Kg) | disease | stage | Therapies | Background | information |

Patients

| | | | | | | | | |
|---|---|---|---|---|---|---|---|---|
| HU19 | M | 57 | 100 | Nephrosclerosis | 3A | Lithium, | HTN, | |
| | | | | | | Allopurinol, | Gout, | |
| | | | | | | Venlafaxine, | RCC | |
| | | | | | | Lamotrigine, | Papillary | |
| | | | | | | Lercanidipine, | Type 1, | |
| | | | | | | Minoxidil, | Bipolar | |
| | | | | | | Atenolol, | Disorder | |
| | | | | | | Ropinirole, | | |
| | | | | | | Spironolactone, | | |
| | | | | | | Topiramate, | | |
| | | | | | | VITAMIN D | | |
| HU20 | M | 57 | 73 | Chronic | G5 | Allopurinol, | HTN, | RNA |
| | | | | glomerulonephritis | A2 | Amlodipine, | Gout, | sequencing |
| | | | | | | EPO, | Dyslipidemia, | of urine |
| | | | | | | Doxazosin, | Hepatitis B | derived |
| | | | | | | Aspirin, | (latent), | spheres |
| | | | | | | Atorvastatin, | s/p | |
| | | | | | | Atenolol, | Colorectal | |
| | | | | | | Sodium | cancer | |
| | | | | | | bicarbonate | | |
| HU21 | M | 65 | 94 | Nephrosclerosis/ | G4 | Allopurinol, | Ischemic | RNA |
| | | | | FSGS | | Vitamin D, | Heart | sequencing |
| | | | | | | Ezetimibe + | Disease, | of urine |
| | | | | | | Atorvastatin, | HTN, | derived |
| | | | | | | Levothyroxine, | Dyslipidemia, | spheres |
| | | | | | | Folic acid, | Obstructive | |
| | | | | | | Lercanidipine, | sleep apnea, | |
| | | | | | | Esomeprazole, | Gout, | |
| | | | | | | Clopidogrel, | Hypothyroidism, | |
| | | | | | | Labetalol, | Hepatitis B | |
| | | | | | | Alfuzosin | (latent) | |
| | | | | | | Hydrochloride | | |
| HU22 | M | 9 | | Pearson | 4 | NaCl, Co- | | |
| | | | | syndrome, | | Q10, Alpha | | |
| | | | | Renal | | lipoic acid, | | |
| | | | | Fanconi, | | Calcium, GH | | |
| HU23 | M | 32 | | Proximal | 3A | Sodium | Carnitine | RNA |
| | | | | tubulopathy | | Bicarbonate, | deficiency, | sequencing |
| | | | | | | Potassium | short | of urine |
| | | | | | | Phosphate, | stature, | derived |
| | | | | | | Sodium | Osteomalacia, | spheres |
| | | | | | | Phosphate, | Epilepsy | |
| | | | | | | Multivitamin, | | |
| | | | | | | Iron, Vitamin | | |
| | | | | | | D, L- | | |
| | | | | | | Carnitine, | | |
| | | | | | | Carbamazepine, | | |
| | | | | | | Pregabalin | | |
| HU24 | M | 17 | 75 | Renal | 4 | Enalapril, | Short | |
| | | | | Dysplasia, | | EPO, Iron, | stature | |
| | | | | Nephronophthisis | | Folic Acid, | | |
| | | | | | | GH | | |
| HU25 | F | 36 | 67.3 | IgA | 4 | Brentuximab, | Hodgkin's | |
| | | | | nephropathy | | Calcium, | Lymphoma, | |
| | | | | | | Vitamin D, | Liver | |
| | | | | | | Losartan, | transplantation | |
| | | | | | | Prednisone | | |
| HU27 | M | 11 | 13 | Pearson | 4 | Calcium, | s/p | |
| | | | | syndrome, | | Vitamin D, | Acute | |
| | | | | Renal | | Probiotics, | pancreatitis | |
| | | | | Fanconi | | Creon, | | |
| | | | | | | Sodium | | |
| | | | | | | Citrate, | | |
| | | | | | | Magnesium, | | |
| | | | | | | Sodium | | |
| | | | | | | Phosphate, | | |
| | | | | | | Potassium | | |
| | | | | | | Phosphate, | | |
| | | | | | | Sodium | | |
| | | | | | | Citrate, EPO, | | |
| | | | | | | Carnitine, | | |
| | | | | | | Ferrous | | |
| | | | | | | sulfate, | | |
| | | | | | | Albuterol | | |
| | | | | | | INH, | | |
| | | | | | | Leucovorin, | | |
| | | | | | | CoQ10, | | |
| | | | | | | sodium | | |
| | | | | | | polystyrene | | |
| | | | | | | sulfonate | | |
| HU28 | M | 1 | 11 | Renal | 2 | Fludrocortisone, | | |
| | | | | Tubular | | Iron, Sodium | | |
| | | | | Dysplasia | | bicarbonate | | |
| HU29 | M | 13 | 59 | Renal | 4-5 | Fludrocortisone, | | |
| | | | | Tubular | | Vitamin D, | | |
| | | | | Dysplasia | | Erythropoietin, | | |
| | | | | | | Iron, Sodium | | |
| | | | | | | bicarbonate, | | |
| | | | | | | GH | | |
| HU32 | F | 5 | 14.8 | Pearson | 1 | Calcium, | | |
| | | | | syndrome, | | Magnesium, | | |
| | | | | Renal | | Sodium | | |
| | | | | Fanconi | | Phosphate, | | |
| | | | | | | Potassium | | |
| | | | | | | Citrate | | |
| H35 | M | 12 | 15.9 | Pearson | 1 | Calcium, | | |
| | | | | syndrome, | | Vitamin D, | | |
| | | | | Renal | | Sodium | | |
| | | | | Fanconi | | Citrate, | | |
| | | | | | | Potassium | | |
| | | | | | | Citrate, | | |
| | | | | | | Sodium | | |
| | | | | | | Citrate, | | |
| | | | | | | Sodium | | |
| | | | | | | Phosphate, | | |
| | | | | | | Magnesium | | |

### Example 3

### NRG1 or CD40L Enhance the Growth of UD-EpC

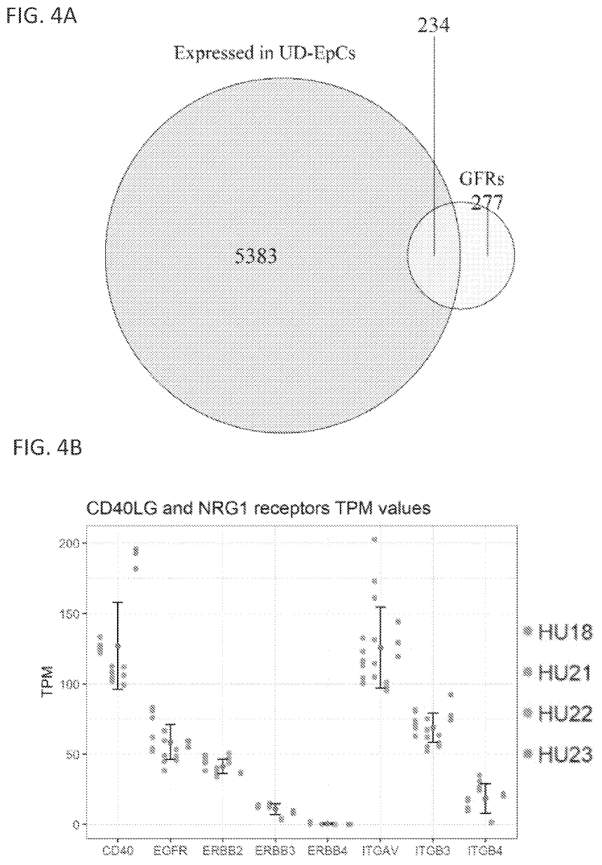

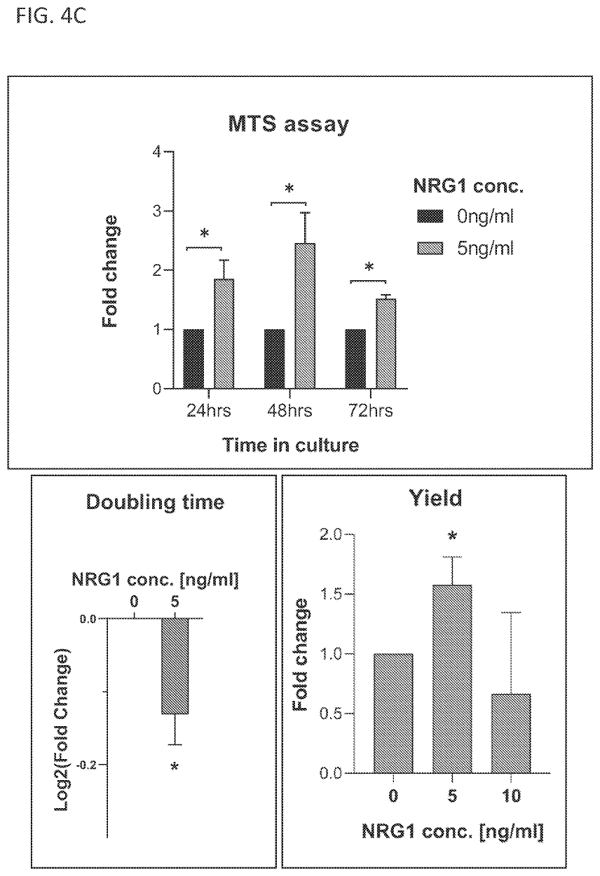

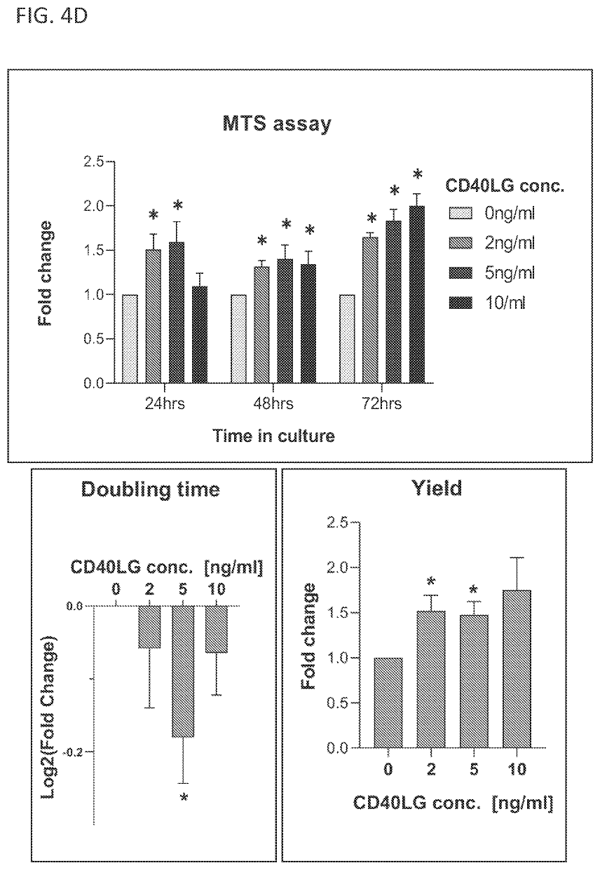

Next, culture conditions were modified so as to augment the proliferation of UD-EpC. To this end, cells were analyzed via RNA-sequencing to thereby identify growth factor receptor genes (according to Uniprot) included in the top 10% most highly expressed genes according to average gene expression values TPM (transcript per million) (FIG. 4A). Out of the 5,383 genes expressed genes in UD-EpC, 234 were growth factor receptors (FIG. 4A). Among the latter, the receptors for NRG1 and CD40L were significantly over-expressed (FIG. 4B). Indeed, the addition of either NRG1 or CD40L to the growth medium at concentrations of 5 ng/ml significantly enhanced cell viability, as determined via the MTS (Methyltetrazolium salt proliferation assay) assay, reduced the doubling time and resulted in a higher cell yield (FIG. 4C-4D).

### Example 4

### UD-EpC are Capable of Generating nSPH

The next step was to determine whether UD-EpC could generate nSPH. For this purpose, the cells were harvested and grown as monolayer in either REMC or KSFM, and seeded in low attachment conditions in SFM. UD-EpC successfully formed nSPH (UD-nSPH), regardless of the medium in which they were grown (FIG. 5A). Notably, nSPH were successfully derived from UD-EpC of both healthy donors and CKD patients (FIG. 5A). Immunostaining demonstrated that all types of UD-nSPH express the epithelial marker cytokeratin (FIG. 5B) and the kidney segment specific markers CD13 (proximal tubule) and EMA (distal tubule), indicating that they represent genuine nSPH, harboring tubular epithelial cells of different lineages (FIG. 5B). Accordingly, characterization via flow cytometry demonstrated that the vast majority of cells within UD-nSPH grown in the two types of media, express the epithelial marker EpCAM, and that both proximal tubular CD13⁺ and, to a lesser extent, distal tubular EMA⁺ cells are present within the nSPH (FIG. 5C). Notably, UD-nSPH derived from CKD patients with tubular and glomerular disorders exhibited similar expression levels of these markers. Taken together, these results indicate that UD-EpC from both healthy individuals and CKD patients are capable of generating proliferative nSPH.

### Example 5

### UD-EpC Grown as UD-nSPH Undergo Mesenchymal to Epithelial Transition (MET) with Widespread Activation of Tubular Epithelial Markers

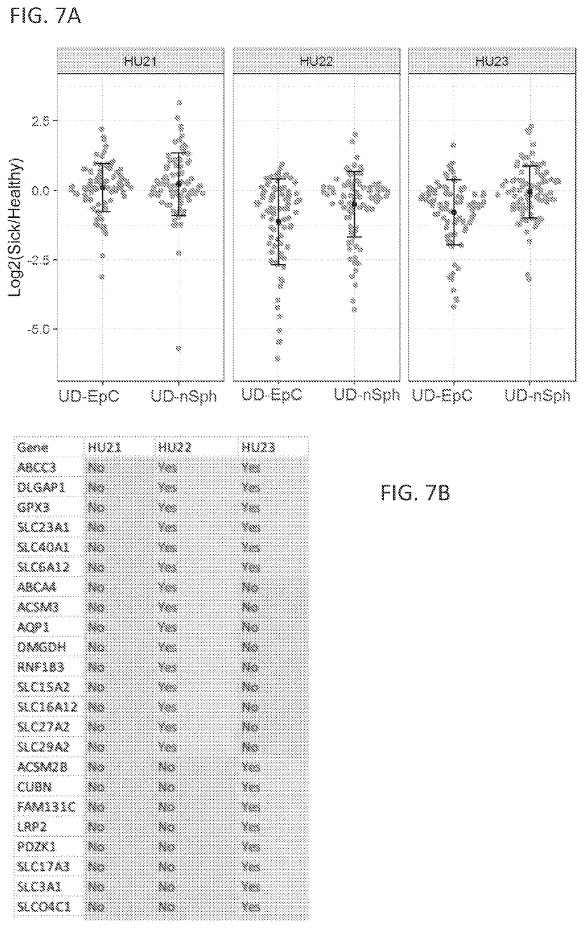

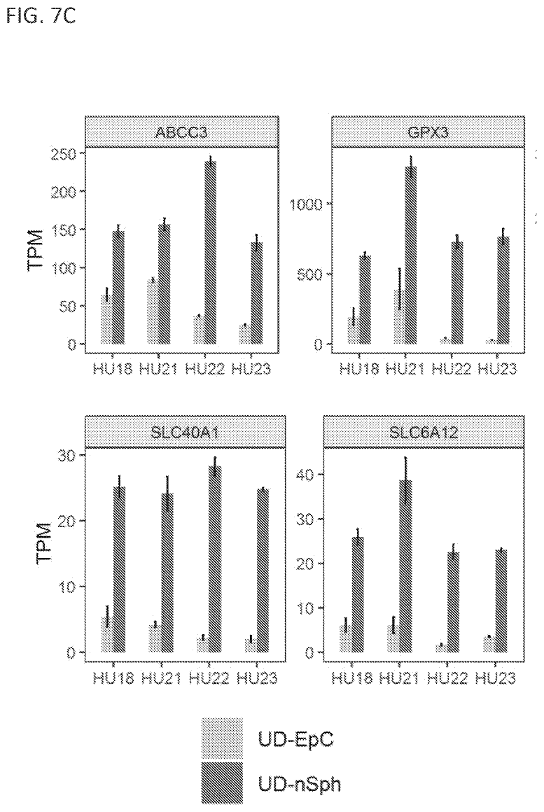

Next, global gene expression analysis of UD-nSPH was done by comparing them to the 2D UD-EpC from which they were derived, via RNA-sequencing, using 2D cultures and nSPH obtained from one healthy donor and three CKD patients, each grown in either REMC or KSFM. Euclidean distance analysis and principal component analysis (PCA) demonstrated that the most significant determinant of transcriptional similarity is the culturing method (i.e. adherent vs. nSPH), followed by donor and growth medium, with the latter having little effect on gene expression. (FIG. 6A-6B). Volcano plot representation of differentially expressed genes in UD-nSPH compared to UD-EpC demonstrated significant inhibition of epithelial-mesenchymal transition (EMT), as evident by down-regulation of SNAI2, SNAI1 and VIM, alongside up-regulation of CDH1, CLDN2 and GRHL2 in UD-nSPH (FIG. 6C). Accordingly, gene set enrichment analysis (GSEA) comparing UD-nSPH and UD-EpC indicated in the former activation of the Wnt signaling pathway and inhibition of cell cycle progression and EMT (FIG. 6D). Consistent with the inhibition of EMT in UD-nSPH, widespread activation of genes encoding for renal tubular epithelial cell types was indicated (FIG. 6E). Taken together, these results demonstrate that UD-nSPH represent renal epithelial structures recapitulating the gene expression of native kidneys. Next, the present inventors compared cells derived from healthy individuals to those derived from CKD patients. Donor HU21 is suffering from nephrosclerosis which mostly defined by damage to the glomeruli while donors HU22 and HU23 both have damage in their proximal tubule. When the expression levels of proximal tubule marker was compared between healthy donor (HU18) and sick donor HU21 there was no differences in both UD-EpC and UD-nSPH (FIG. 7A). However, donors HU22 and 23 showed down-regulation in these set of genes in UD-EpC (FIG. 7A). Surprisingly, the expression of proximal epithelial gene were corrected when cells were grown in 3D conditions, compared to healthy donor (FIG. 7A). Closer look on specific genes demonstrate the differences between glomerular and tubular injured CKD patients (FIGS. 7B and 7C). Next the present inventors compared UD-EpC to KD-EpC (kidney derived epithelial cells) in both culture conditions (2D and 3D). Differentially expressed genes (DE) were compared between nSPH (3D) and EpC (2D) in both UD and KD cells. Venn diagram showed overlap between the samples in both up and down regulated DE genes confirming that UD cultures composed of kidney cells (FIG. 8A). However, the present inventors identified groups of genes unique to each method of cell collection (urine or kidney) (FIG. 8A). Next Pearson correlation analysis was performed between RNA-seq data of UD-EpC and KD-EpC in both culture conditions. As expected, a correlation was found between cells originated from kidney tissue or from urine between different donors in both culture conditions (FIG. 8B). Cells cultured in 2D and 3D showed high correlation coefficient when derived from kidney tissue, and less correlative when derived from urine (FIG. 8B). Low correlation was detected between two cell origins indicated variance in the expression pattern of the samples (FIG. 8B). Furthermore, higher expression levels in renal segment epithelial genes between cells that are urine origin and tissue origin in both culture conditions (FIG. 8C), indicating that the process of isolating kidney cells from urine is different from isolating kidney from tissue. It can be concluded that even though the transcriptome of the UD-EpC may differ between patients with different kidney diseases, when the cells are cultured as UD-SPH they represented similar transcriptome. FIG. 8A-C show the differences between cells originated from tissue and cells originated from urine.

### Example 6

### UD-nSPH Express the SARS-CoV-2 Entry Receptors and are Susceptible to HCoV-229E Infection

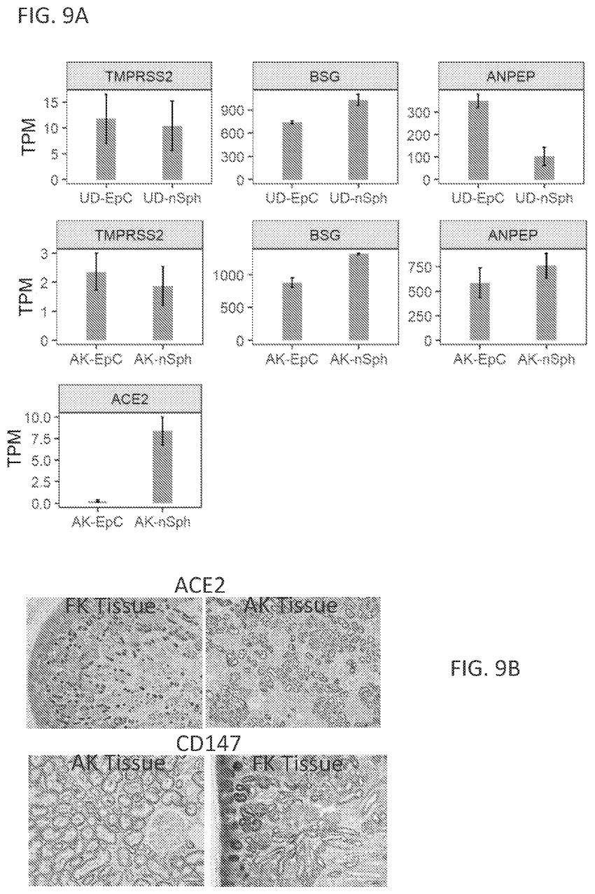

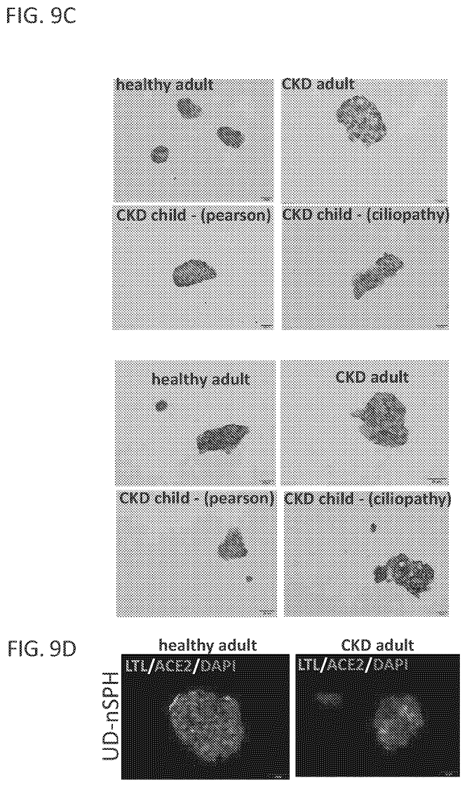

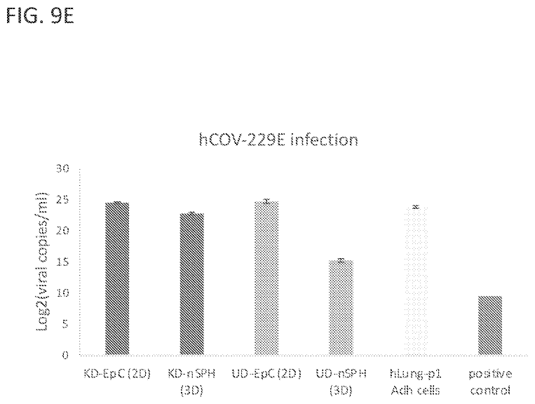

Having shown that UD-nSPH recapitulate human kidneys, the present inventors were next interested in assessing whether they could serve as a model to study the host-pathogen interaction of human cells and the SARS-CoV-2, which often affects the kidneys and has been shown to directly infect renal tubular cells²⁶. First, the present inventors assessed whether UD-nSPH express the surface markers thought to serve as receptors for SARS-CoV-2, ACE2, TMPRSS2 and BSG (CD147)^{21,22}. Both KD-nSPH and UD-nSPH expressed similar or higher levels of TMPRSS2 and CD147 in comparison to their 2D culture counterparts (FIG. 9A), whereas only KD-nSPH, but not UD-nSPH expressed ACE2. Notably, both types of nSPH expressed ANPEP (CD13), which is the cell entry receptor for the HCoV-229E, a strain of coronavirus that is a common cause of common cold. Immunostaining of both adult and fetal human kidneys confirmed robust and widespread tubular expression of ACE2 and CD147 at the protein level (FIG. 9B). Interestingly, despite the low levels of expression of the ACE2 transcript, UD-nSPH exhibited strong expression of both ACE2 and CD147 at the protein level, regardless of whether the donor was healthy or a CKD patient (FIGS. 9C-9D). Finally, as a proof of concept, and given the expression of its receptor in UD-nSPH, the present inventors were interested in determining whether UD-nSPH could be infected with the HCoV-229E strain. For this purpose, KD-EpC, KD-nSPH, UD-EpC and UD-nSPH were incubated with HCoV-229E for 48 hours and the level of infection was assessed by the viral copies in the cells. Primary passage 1 lung cells served as positive control. Quantitative real-time PCR demonstrated high levels of viral load within all cell types, with KD-EpC, KD-nSPH and UD-EpC demonstrating comparable levels of infection as human lung cells (FIG. 9E). Interestingly, UD-nSPH, which exhibited lower expression of the viral receptor ANPEP, also demonstrated lower levels of infection (FIG. 9E). Taken together, these results indicate that UD-nSPH express the receptors for SARS-CoV-2 and HCoV-229E, and are readily infected by the latter, with infection efficacy correlating with the receptor's expression levels.

### Example 7

### UD-nSPH are Susceptible to SARS-COV2 Infection

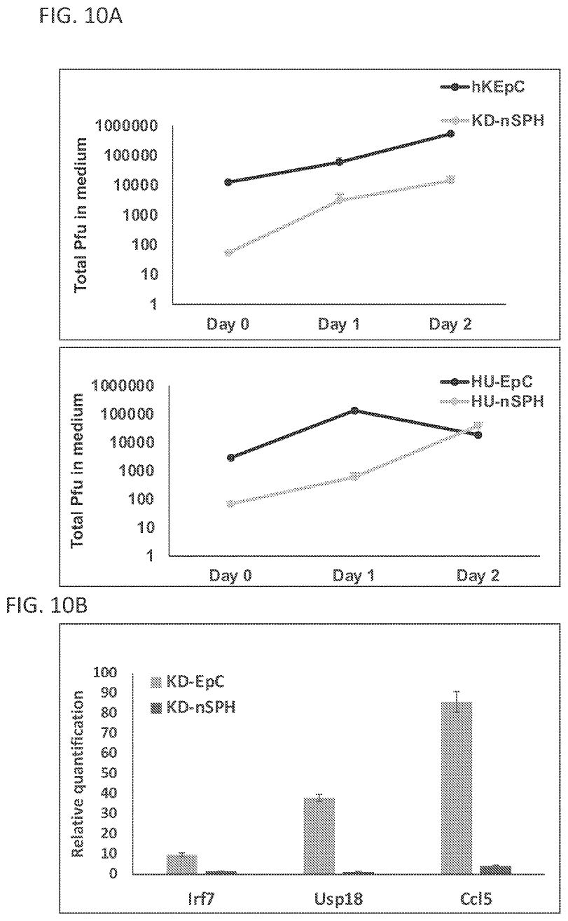

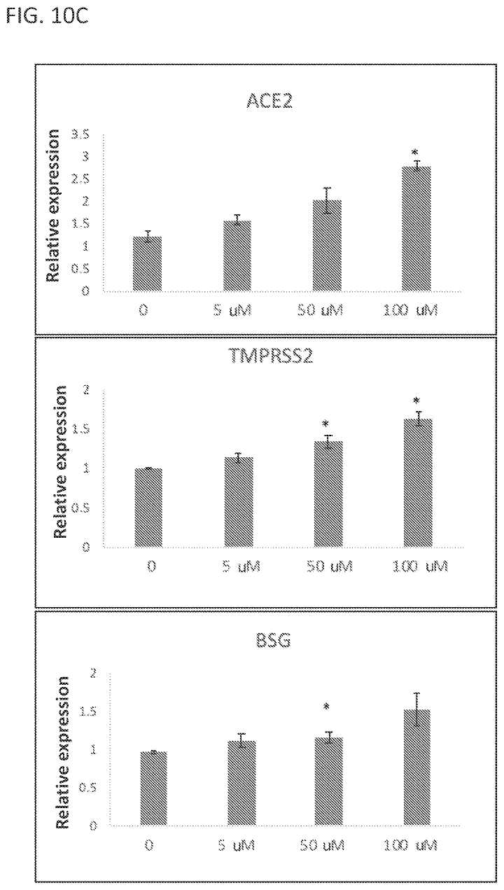

Having shown that kidney cells effectively facilitate infection of HCoV-229E, we next attempted to infect both types of cultures with SARS-CoV-2, quantifying the level of infection according to the number of plaque forming units equivalents per ml (pfuE/ml) of medium as measured by qPCR at days 0, 1, and 2 (FIG. 10A). Interestingly, IRF7, USP18 and CCL5, genes which are activated during viral infection²⁷⁻²⁹, were induced in hKEpCs and kSPH indicating active infection (FIG. 10B). Taken together, these results indicate that while human kidney cells are susceptible to infection by SARS-CoV-2. Lastly, we were interested in testing the relevance of the kSPH as a model for assessing drug-related effects that might be of importance in COVID-19. We asked whether ACE inhibitors affect the expression of SARS-CoV-2 receptors, including ACE2, a highly debated question that may influence susceptibility to infection by SARS-CoV-2, but has nonetheless yielded conflicting results in animal models³⁰⁻³³. Toward this end, we treated kSPH with increasing doses of the ACE inhibitor Ramipril for 48 hours and used qPCR to test the effect on the expression of ACE2, TMPRSS2, and BSG. We found that Ramipril treatment resulted in a significant induction of all three receptors, particularly ACE2 demonstrating a dose-response relationship (FIG. 10C). Taken together, these results suggest that ACE inhibitors lead to upregulation of the SARS-CoV-2 receptors in human cells, which in turn might increase patient susceptibility to infection.

### Example 8

### UD-nSPH Harbor Renal Tubulogenic Potential

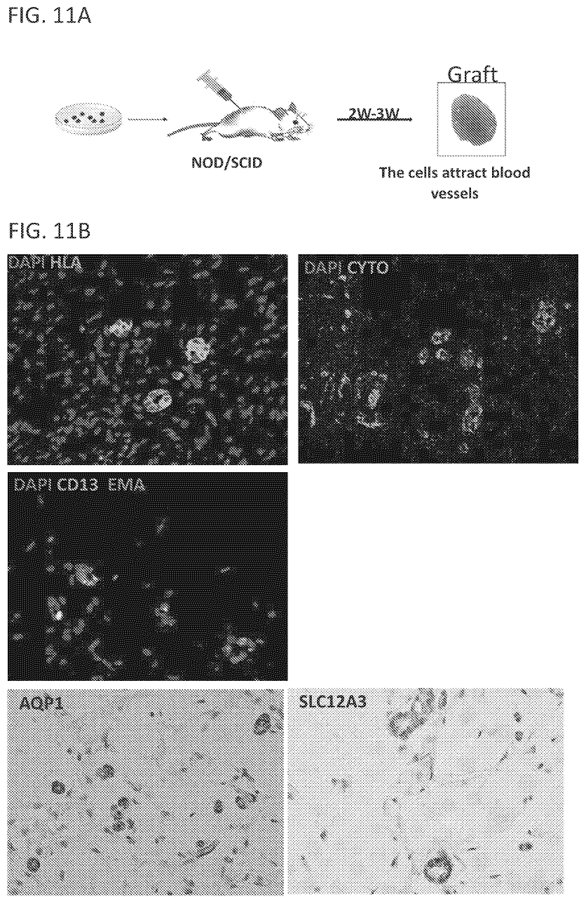

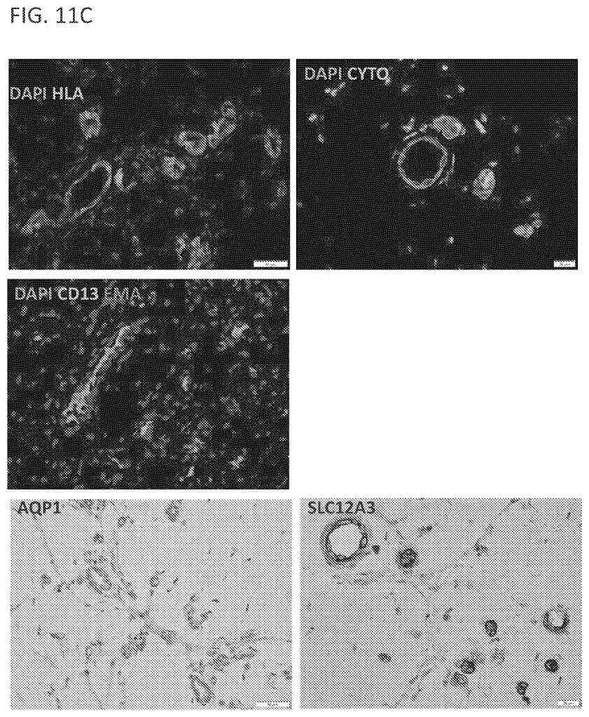

Having shown that UD-EpC can generate nSPH, the present inventors were interested in determining whether the latter are capable of giving rise to renal epithelial structures. Toward this aim, UD-nSPH were injected subcutaneously into NOD-SCID mice within Matrigel, to assess their in-vivo tubulogenic capacity⁸. Following 14-21 days, the grafts were removed for histological analysis (FIG. 11A). Staining of UD-nSPH-derived grafts for the human specific marker HLA revealed the presence of tubular structures (FIG. 11B), some of which exhibited clear lumens, derived from the injected cells. Upon staining the grafts for the proximal tubule markers CD13 and AQP1, and the distal tubule markers EMA and SLC12a3 (FIG. 11B), the presence of both markers was detected. Taken together, these results indicate that UD-nSPH harbor multipotential tubulogenic capacity in-vivo.

### Example 9

### Intra-Renal Administration of Urine-Derived kSPH Results in Long-Term Engraftment, Differentiation and Self-Organization into Tubular Structures and Reduction of Kidney Fibrosis

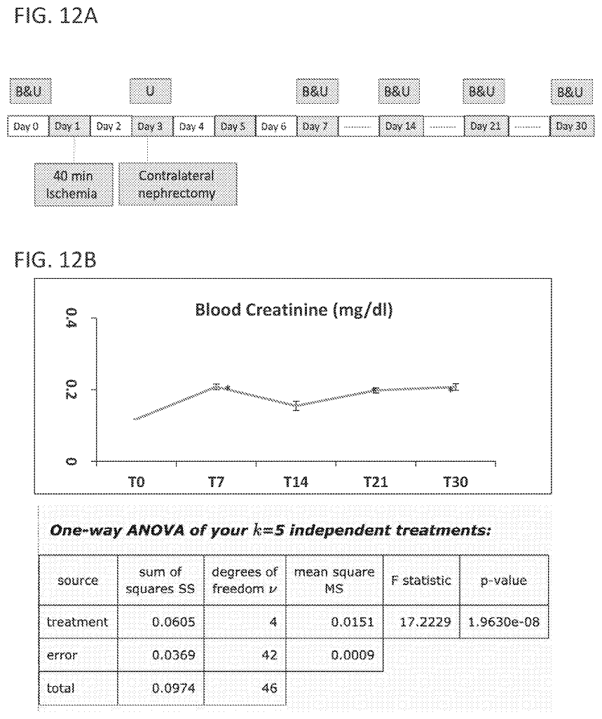

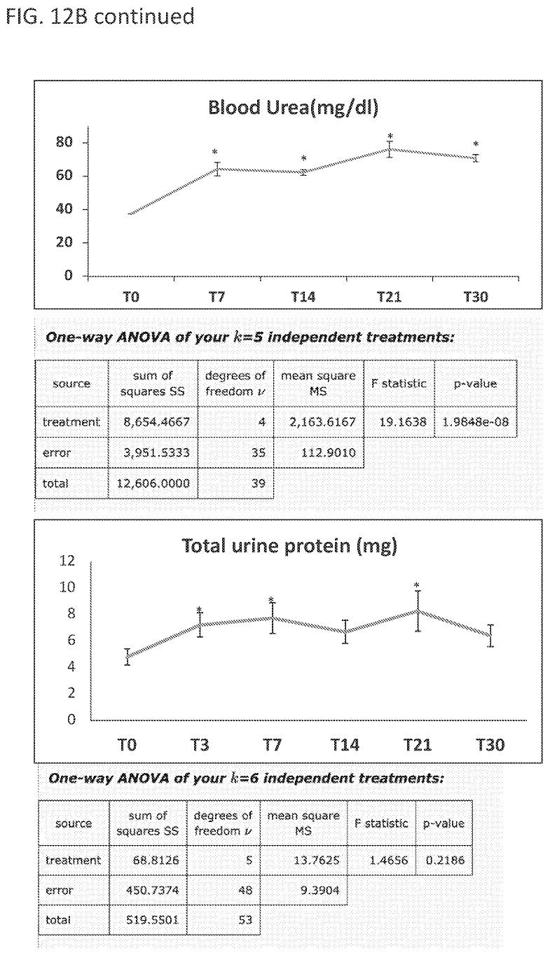

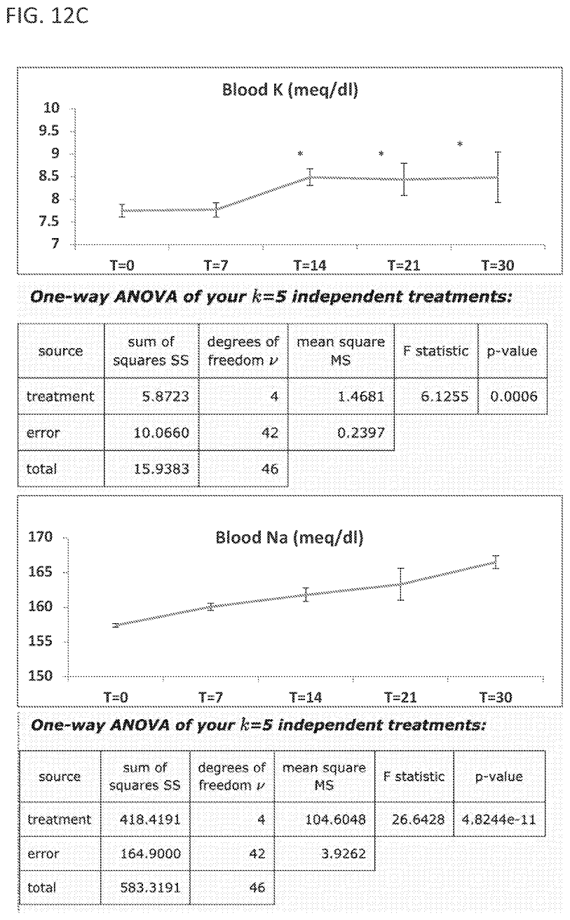

In order to assess the therapeutic potential of UD-nSPH, the present inventors established a new CKD model in mice. The new model begins with 40 minutes of ischemia to one kidney and continues 3 days later with the resection of the contralateral kidney (FIG. 12A). Although mice can recover from ischemic kidney damage (that simulates acute kidney injury), the nephrectomy of the contralateral kidney prevents such recovery, hence causing deterioration to CKD.

To ensure that the kidney was damaged, the present inventors collected 24 hr urine samples using metabolic cages and retrieved blood from the orbital sinus as shown in the scheme (FIG. 12A). The results showed increased blood creatinine and urea levels over time (T=7,14,21,30 days) compared to baseline (T=0), alongside increased levels of total urinary protein), all indicating compromised kidney function (FIG. 12B). Analysis of blood electrolytes (potassium [K] and Sodium [Na]) also supported this conclusion (FIG. 12C).

At day 35, the mice were administered with UD-nSPH by direct injection into the kidney parenchyma (treatment group). The control group included mice that underwent the same procedure using a syringe without cells. After 3 weeks, the mice were injected for the second time and 3 weeks later the mice were sacrificed, and their kidney was harvested for histology FIG. 13A). Next, the injected kidneys were stained for the human marker HLA to detect allogeneic engraftment. As shown, the cells could engraft in the mouse kidney tissue and could organize in tubular structures (FIG. 13B). Further analysis showed that the human tubular structures that were formed stained for the segment specific markers EMA and CD13 (markers of distal and proximal tubules, respectively) indicating renal identity (FIG. 13B). Notably, the fact that the cells have engraftment capacity on top of their tubulogenic potential is highly important from the regenerative perspective, signifying their potential ability to increase the relative proportion of epithelial tissue within fibrotic kidneys. To test this hypothesis, the present inventors examined tissue fibrosis levels using Masson-Trichrome staining in treated (injected mice) and untreated (control) mouse tissues (FIG. 13C). Indeed, it was found that treated mice have significantly less fibrotic connective tissue (blue staining) than the control group (FIG. 13C).

### Example 10

### Urine-Derived kSPH Show Anti-Fibrotic Effect when Co-Cultured with Fibroblasts: The Role of the Secretome and its Composition

Next the analysis of the anti-fibrotic effects of UD-nSPH was elaborated using co-culture experiments in which UD-nSPH or UD-EpC were placed in proximity to fibroblasts, but not allowing direct contact (FIG. 14A). UD-nSPH actively inhibited the production of fibrosis-related genes, in fibroblasts, including collagen molecules (COL1A1, COL1A2, COL3A1 and PERIOSTIN) indicating a paracrine mechanism of action (FIG. 14B). Importantly, similar co-culture experiments using UD-EpC and fibroblasts showed elevation in expression of fibrosis related genes (FIG. 14C). This suggested that UD-nSPH harbor an advantage in inhibiting fibrosis-related genes in a paracrine manner. Transcriptomic data comparing UD-nSPH to their adherent counterparts showed upregulation in kidney epithelial markers, fatty oxidation and oxidative phosphorylation, activation of MET genes and inhibition of EMT genes all indicating a shift from pro-fibrotic, pro-inflammatory to an anti-fibrotic, anti-inflammatory gene signature set in UD-nSPH (FIG. 14D).

To further interrogate that anti-fibrotic effect of UD-nSPH, the present inventors explored the protein composition of the UD-nSPH secretome compared to UD-EpC using proteomics. UD-nPSH secretome showed elevation in reno-protective proteins (FGL2 and LTF) and antifibrotic proteins (APOE, CDH1 and VDAC1) (FIG. 14E) along with downregulation in fibrosis related proteins (TGFb, COL1A1, CTGF and FN1) (FIG. 1E).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

1. www(dot)cdc(dot)gov/diabetes/pubs/pdf/kidney_factsheet.pdf.
2. www(dot)kidney(dot)org/news/newsroom/factsheets/FastFacts. 2016.
3. Pleniceanu O, Harari-Steinberg O, Dekel B. Concise review: Kidney stem/progenitor cells: differentiate, sort out, or reprogram? Stem Cells. September 2010; 28(9):1649-1660.
4. Dekel B. The Ever-Expanding Kidney Repair Shop. J Am Soc Nephrol. June 2016; 27(6):1579-1581.
5. Wu H, Uchimura K, Donnelly E L, Kirita Y, Morris S A, Humphreys B D. Comparative Analysis and Refinement of Human PSC-Derived Kidney Organoid Differentiation with Single-Cell Transcriptomics. Cell Stem Cell. Dec. 6 2018; 23(6):869-881 e868.
6. Rinkevich Y, Montoro D T, Contreras-Trujillo H, et al. In vivo clonal analysis reveals lineage-restricted progenitor characteristics in mammalian kidney development, maintenance, and regeneration. Cell Rep. May 22 2014; 7(4):1270-1283.
7. Buzhor E, Harari-Steinberg O, Omer D, et al. Kidney spheroids recapitulate tubular organoids leading to enhanced tubulogenic potency of human kidney-derived cells. Tissue Eng Part A. September 2011; 17(17-18):2305-2319.
8. Harari-Steinberg O, Omer D, Gnatek Y, et al. Ex Vivo Expanded 3D Human Kidney Spheres Engraft Long Term and Repair Chronic Renal Injury in Mice. Cell Rep. Jan. 21 2020; 30(3):852-869 e854.
9. Linder D. Culture of cells from the urine and bladder washings of adults. Somatic cell genetics. May 1976; 2(3):281-283.
10. Felix J S, Littlefield J W. Urinary tract epithelial cells cultured from human urine. International review of cytology. Supplement. 1979(10):11-23.
11. Bharadwaj S, Liu G, Shi Y, et al. Characterization of urine-derived stem cells obtained from upper urinary tract for use in cell-based urological tissue engineering. Tissue Eng Part A. August 2011; 17(15-16):2123-2132.
12. Zhang Y, McNeill E, Tian H, et al. Urine derived cells are a potential source for urological tissue reconstruction. J Urol. November 2008; 180(5):2226-2233.
13. Dekel B, Zangi L, Shezen E, et al. Isolation and characterization of nontubular sca-1+lin-multipotent stem/progenitor cells from adult mouse kidney. J Am Soc Nephrol. December 2006; 17(12):3300-3314.
14. Pleniceanu O, Shukrun R, Omer D, et al. Peroxisome proliferator-activated receptor gamma (PPARgamma) is central to the initiation and propagation of human angiomyolipoma, suggesting its potential as a therapeutic target. EMBO Mol Med. April 2017; 9(4):508-530.
15. Pleniceanu O, Omer D, Harari-Steinberg O, Dekel B. Renal lineage cells as a source for renal regeneration. Pediatr Res. January 2018; 83(1-2):267-274.
16. Humphreys B D, Valerius M T, Kobayashi A, et al. Intrinsic epithelial cells repair the kidney after injury. Cell Stem Cell. Mar. 6 2008; 2(3):284-291.
17. Inoue C N, Kondo Y, Ohnuma S, Morimoto T, Nishio T, Iinuma K. Use of cultured tubular cells isolated from human urine for investigation of renal transporter. Clin Nephrol. February 2000; 53(2):90-98.
18. Rahmoune H, Thompson P W, Ward J M, Smith C D, Hong G, Brown J. Glucose transporters in human renal proximal tubular cells isolated from the urine of patients with non-insulin-dependent diabetes. Diabetes. December 2005; 54(12):3427-3434.
19. World Health Organization; WHO Director-General's opening remarks at the media briefing on COVID-19-3 Mar. 2020. 3 Mar. 2020.
20. Walls A C, Park Y J, Tortorici M A, Wall A, McGuire A T, Veesler D. Structure, Function, and Antigenicity of the SARS-CoV-2 Spike Glycoprotein. Cell. Mar. 6 2020.
21. Hoffmann M, Kleine-Weber H, Schroeder S, et al. SARS-CoV-2 Cell Entry Depends on ACE2 and TMPRSS2 and Is Blocked by a Clinically Proven Protease Inhibitor. Cell. Apr. 16 2020; 181(2):271-280 e278.
22. Wang K, Chen W, Zhou Y-S, et al. SARS-CoV-2 invades host cells via a novel route: CD147-spike protein. bioRxiv. 2020:2020.2003.2014.988345.
23. Lely A T, Hamming I, van Goor H, Navis G J. Renal ACE2 expression in human kidney disease. J Pathol. December 2004; 204(5):587-593.
24. Prescott L F. The normal urinary excretion rates of renal tubular cells, leucocytes and red blood cells. Clinical science. December 1966; 31(3):425-435.
25. Felix J S, Sun T T, Littlefield J W. Human epithelial cells cultured from urine: growth properties and keratin staining. In vitro. October 1980; 16(10):866-874.
26. Farkash E A, Wilson A M, Jentzen J M. Ultrastructural Evidence for Direct Renal Infection with SARS-CoV-2. J Am Soc Nephrol. May 5 2020.
27. Au W C, Moore P A, LaFleur D W, Tombal B, Pitha P M. Characterization of the interferon regulatory factor-7 and its potential role in the transcription activation of interferon A genes. J Biol Chem. Oct. 30 1998; 273(44):29210-29217.
28. Crawford A, Angelosanto J M, Nadwodny K L, Blackburn S D, Wherry E J. A role for the chemokine RANTES in regulating CD8 T cell responses during chronic viral infection. PLoS pathogens. July 2011; 7(7):e1002098.
29. Ritchie K J, Hahn C S, Kim K I, et al. Role of ISG15 protease UBP43 (USP18) in innate immunity to viral infection. Nat Med. December 2004; 10(12):1374-1378.
30. Ferrario C M, Jessup J, Gallagher P E, et al. Effects of renin-angiotensin system blockade on renal angiotensin-(1-7) forming enzymes and receptors. Kidney Int. November 2005; 68(5):2189-2196.
31. Jessup J A, Gallagher P E, Averill D B, et al. Effect of angiotensin II blockade on a new congenic model of hypertension derived from transgenic Ren-2 rats. American journal of physiology. Heart and circulatory physiology. November 2006; 291(5):H2166-2172.
32. Li Y, Zeng Z, Li Y, et al. Angiotensin-converting enzyme inhibition attenuates lipopolysaccharide-induced lung injury by regulating the balance between angiotensin-converting enzyme and angiotensin-converting enzyme 2 and inhibiting mitogen-activated protein kinase activation. Shock. April 2015; 43(4):395-404.
33. Wysocki J, Lores E, Ye M, Soler M J, Batlle D. Kidney and Lung ACE2 Expression after an ACE Inhibitor or an Ang II Receptor Blocker: Implications for COVID-19. J Am Soc Nephrol. Jul. 15 2020.

What is claimed is:

1. A method of expanding kidney epithelial cells, the method comprising:
(a) isolating cells from urine of a subject;
(b) culturing said cells under adherent conditions, so as to obtain urine-derived epithelial cells (UD-EpC);
(c) passaging said UD-EpC.

2. The method of claim 1, wherein said culturing is performed in the presence of serum.

3. The method of claim 1, wherein said culturing is performed in RE:MC.

4. The method of any one of claims 1-3, wherein said culturing is performed in the presence of CD40 ligand (CD40L), e.g., (2-10 ng/ml each).

5. The method of any one of claims 1-3, wherein said culturing is performed in the presence of neuregulin-1 (NRG1), e.g., (5-10 ng/ml).

6. The method of any one of claims 1-5, wherein said culturing is performed in the presence of isolated mitochondria.

7. The method of any one of claims 1-3, wherein said isolating is by centrifugation.

8. The method of any one of claims 1-7, wherein said adherent conditions comprise gelatin coating.

9. The method of any one of claims 1-8, wherein said subject is a male subject.

10. The method of any one of claims 1-8, wherein said subject is a female subject.

11. The method of claim 1, wherein said passaging is performed to enrich UD-EpC and deplete squamous epithelial cells of vagina and/or bladder origin.

12. The method of any one of claims 1-11, wherein said subject is a healthy subject.

13. The method of any one of claims 1-11, wherein said subject is diagnosed with a kidney disease.

14. The method of any one of claims 1-13, wherein said kidney disease is a chronic kidney disease (CKD).

15. The method of claim 14, wherein said subject is a human subject.

16. The method of claim 15, wherein said human subject is an adult.

17. The method of any one of claims 1-14, wherein said UD-EpC express Ace2.

18. The method of any one of claims 1-17, wherein said UD-EpC are CD13+/EMA+/EpCAM+.

19. A culture comprising the UD-EpC obtainable according to the method of any one of claims 1-18.

20. A method of producing a nephrospheroid, the method comprising culturing the UD-EpC of the method of any one of claims 1-18 under non-adherent conditions, thereby generating the nephrospheroid.

21. The method of claim 20, wherein said nephrospheroid is capable of forming a tubular nephric tissue upon transplantation.

22. The method of any one of claims 20-21, wherein said nephrospheroid is capable of generating a proximal tubule compartment.

23. The method of claim 22, wherein said proximal tubule compartment expresses Ace2.

24. The method of any one of claims 20-23, wherein said nephrospheroid is capable of generating a distal tubule compartment.

25. The method of any one of claims 20-24, wherein said nephrospheroid is CD13+/EMA+/EpCAM+/Ace2+ at the protein level and CD13+/EMA+/EpCAM+/Ace2− at the RNA level.

26. A nephrospheroid obtainable according to the method of any one of claims 20-24.

27. The nephrospheroid of claim 26, characterized by gene expression as in FIG. 8C (human urine), e.g., higher expression of ATP12A, ACMS2A and/or SLC16A7 than that derived from human kidney.

28. The nephrospheroid of any one of claims 26 and 27 having an anti-fibrotic activity.

29. A nephrospheroid comprising urine-derived epithelial cells, the nephrospheroid is capable of forming a tubular nephric tissue upon transplantation and/or having an anti-fibrotic activity.

30. Cells or secretome of the nephrospheroid of any one of claims 26-29.

31. A method of regenerating renal function, the method comprising administering to a subject in need thereof the nephrospheroid of any one of claims 26-29 or cells or secretome of claim 30, thereby regenerating renal function.

32. A method of drug design, the method comprising determining an effect of a test drug on the nephrospheroid of any one of claims 26-29 or cells or secretome of claim 30.

33. The method of claim 32, wherein said determining is performed in the presence of a Coronavirus.

34. The method of claim 33, wherein said Coronavirus is SARS-CoV-2.

35. A method of analyzing infectivity of a Coronavirus, the method comprising:
(a) contacting a renal epithelial cell culture or a nephrospheroid produced of said culture with a Coronavirus; and
(b) determining infectivity of said Coronavirus in the culture or in the nephrospheroid following said contacting.

36. The method of claim 35, wherein said renal epithelial cell culture is kidney-derived or urine-derived epithelial cells.

37. A method of personalized therapy, the method comprising:
(a) contacting a Coronavirus-infected renal epithelial cell culture or nephrospheroid produced of said culture with a test drug; and
(b) determining an alleviation in viral load following said contacting, said alleviation being indicative of an efficacious therapy.

38. The method of any one of claims 35-37, wherein said renal culture or nephrospheroid is autologous.
